(19) United States
(12) Patent Application Publication
(10) Pub. No.: US 2007/0020237 A1
(43) Pub. Date: Jan. 25, 2007

(54) Glucose inducible insulin expression and methods of treating diabetes

(75) Inventors: Chungja Yoon, Rockville, MD (US); Ji-Won Yoon, Rockville, MD (US); Jaeseok Han, Lake Bluff, IL (US)

Correspondence Address: MCDERMOTT WILL & EMERY LLP;ATTN: INTELLECTUAL PROPERTY DEPTARTMENT, 28 STATE STREET, BOSTON, MA, 02109, US

(73) Assignee: Biotech Institute for International Innovation, Inc.

(21) Appl. No.: 11/443,575

(22) Filed: May 30, 2006

Related U.S. Application Data
(60) Provisional application No. 60/686,797, filed on Jun. 1, 2005.

(51) Int. Cl. C12N 5/08; A61K 48/00; C12N 15/86

(52) U.S. Cl. 424/93.2; 435/456; 435/366

(57) ABSTRACT

The invention provides an isolated tissue specific glucose responsive promoter having a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE). The promoter can include a second or third tripartite transcription factor binding cis element. A host cell including the tissue specific glucose responsive promoter of the invention also are provided. Further provided is a method of treating or preventing diabetes. The method includes administering to an individual an effective amount of a viral particle having a vector comprising a tissue specific glucose responsive promoter comprising a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE) operationally linked to an insulin encoding nucleic acid, wherein expression of the insulin encoding nucleic acid is tissue specific and glucose responsive.

This application is based on, and claims the benefit of, U.S. Provisional Application No. 60/686,797, filed Jun. 1, 2005, entitled “Glucose Inducible Insulin Expression and Methods of Treating Diabetes”, and is incorporated herein by reference.

### BACKGROUND OF THE INVENTION

The present invention relates generally to methods for the treatment and prevention of diabetes and, more specifically, to the regulated expression of insulin for therapeutic treatment of diabetes.

In an individual with normal regulation of blood glucose, the pancreatic hormone insulin is secreted in response to increased blood sugar levels. Increased blood glucose generally occurs following a meal and results in insulin action on peripheral tissues such as skeletal muscle and fat. Insulin stimulates cells of these peripheral tissues to actively take up glucose from the blood and convert it to forms for storage. This process is also referred to as glucose disposal. The levels of blood glucose vary from low to normal to high throughout the day within an individual, depending upon whether the person is in the fasting, intermediate, or fed state. These levels are also referred to as hypoglycemia, euglycemia and hyperglycemia, respectively. In the diabetic individual, these changes in glucose homeostasis are disregulated due to either faulty insulin secretion or action, resulting in a chronic state of hyperglycemia.

Diabetes mellitus is a common disorder, with a prevalence of about 4-5%. The risk of developing diabetes increases with increased weight, with as many as 90% of adult onset diabetic patients being obese. Therefore, due to the high incidence of obese adults, the incidence of adult onset diabetes is increasing worldwide. Diabetes mellitus is classified into three major forms. Type 2 diabetes is one form and is also referred to as non-insulin dependent diabetes (NIDDM) or adult-onset diabetes. Type 1 diabetes is the second form and is referred to as insulin-dependent diabetes (IDDM). The third type of diabetes is genetic and is due to mutations in genes controlling pancreatic islet beta (β) cell function. Although the diagnosis of diabetes is based on glucose measurements, accurate classification of all patients is not always possible. Type 2 diabetes is more common among adults and type 1 diabetes dominates among children and teenagers.

Diabetes mellitus of both types 1 and 2 are associated with a shortened life expectancy as well as other complications such as vascular disease and atherosclerosis. Long-term management of diabetes to prevent late complications often includes insulin therapy regardless of whether the patients are classified as type 1 or type 2. Type 1 diabetes is an auto-immune disease which is associated with near complete loss of the insulin producing pancreatic β cells. This loss of β cells results in insulin-dependence for life. Type 1 diabetes can occur at any age and it has been estimated that about 0.3-1% of all newborns in Caucasian population will develop this disease during their lifetime.

A widely used method of treatment for type 1 diabetes and to some extent type 2 diabetes has classically consisted of insulin maintenance therapy. Such therapy in its simplest form requires the injection of purified or recombinant insulin into a patient following ingestion of a meal or at regular intervals throughout the day to maintain normal blood glucose levels. These injections are required ideally at a frequency of four times per day. Although the above method of treatment provides some benefit to the patient, this method of insulin therapy nevertheless suffers from inadequate blood glucose control as well as requiring a great deal of patient compliance.

Another method of treatment for type 1 diabetes includes the use of devices such as an insulin pump which allows for the scheduled delivery of insulin. This method can be preferable to the method described above due to the need for less frequent injections. However, the use of an insulin pump therapy also has drawbacks in that replacement of a needle once every three days is still required. Similar to insulin maintenance therapy, the insulin pump method also does not achieve optimal glucose regulation as the delivery of insulin is not regulated in response to changes in blood glucose level. These methods of treating diabetes are therefore burdensome as well as inadequate. Furthermore, these methods also have not been completely effective over the course of an average adult lifetime and or have been shown to be effective in preventing this disease.

Various approaches of cell therapy for replacing bioactive insulin into a diabetic individual have been attempted. These include gene therapy approaches, immunotherapies and use of artificial β cells. In vivo gene therapy for the expression of insulin or other polypeptides has included liver targeted viral mediated transduction in animal models. However, these approaches have not provided glucose regulated insulin delivery in therapeutically effective amounts nor have they restored or regenerated insulin producing β cells and, therefore, have seen limited applications in patients.

For example, insulin gene therapy for type 1 diabetes preferably includes an appropriate regulatory system, so that insulin will be produced in response to an increase in blood glucose levels, and be suppressed as blood glucose decreases. Various attempts have been made to achieve glucose-responsive insulin production using naturally occurring glucose-regulated promoters. However, the promoters used were unable to achieve sufficiently high transcriptional activities and at least moderate hyperglycaemia was exhibited in treated animals under non-fasting condition.

Thus, there exists a need for cell-specific expression elements with effective glucose responsiveness and methods that can regulate glucose homeostasis in a diabetic individual in ways that restore the physiological capacity of insulin producing β cells. The present invention satisfies this need and provides related advantages as well.

### SUMMARY OF THE INVENTION

The invention provides an isolated tissue specific glucose responsive promoter having a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE). The promoter can include a second or third tripartite transcription factor binding cis element. A host cell including the tissue specific glucose responsive promoter of the invention also are provided. Further provided is a method of treating or preventing diabetes. The method includes administering to an individual an effective amount of a viral particle having a vector comprising a tissue specific glucose responsive promoter comprising a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE) operationally linked to an insulin encoding nucleic acid, wherein expression of the insulin encoding nucleic acid is tissue specific and glucose responsive.

### BRIEF DESCRIPTION OF THE DRAWINGS

FIG. 1 shows the generation of 3-copy modules of cis-regulatory elements for the construction of synthetic promoter libraries. (A) The original multiple cloning sites (MCS) was replaced with new MCS in the pcDNA3.1 vector, generating pcDNA3-NewMCS. (B) Possible combination of 3 different cis-regulatory elements used for the construction of 3-copy modules. Glucose responsive element (G), C/EBP binding element (C), and HNF-1 binding element (H) were mixed and ligated into the sites of KpnI/BamHI/EcoRV, and EcoRV/EcoRI in pcDNA3-NewMCS in order, generating 3-copy SP-D3.

FIG. 2 shows the generation of synthetic promoter libraries. (A) The original MCS replaced with new MCS in the pGL3-Enhancer vector, generating pGL3E-NewMCS. (B) Schematic diagram of synthetic promoters containing 3-, 6-, and 9-copies of cis-regulatory elements. Proximity region of LPK promoter (−96/+12) relative to transcriptional initiation site) were used as a basal promoter for generation of synthetic promoter-reporter construct. 3-copy mocules from 3-copy SP-D3 were transferred to pLPK(−96/+12)-Luc at KpnI/EcoRI sites, generating 3-copy SP-Luc reporter plasmids. Additional 3-copy modules were inserted in 3-copy SP-Luc in either the same (EcoRI/XhoI) or opposite direction (XhoI/NheI), generating 6-copy SPXE-Luc and 6-copy SPXN-Luc plasmids. Nine-copy SP-Luc plasmids were produced by introduction of additional 3-copy module in 6-copy SPXE-Luc at XhoI/NheI site. The arrow indicates the orientation of 3-copy module. The box with 3 cis-elements represents a single position of the 3-copy module which contains a single element.

FIG. 3 shows comparative luciferase activities of synthetic promoters in vitro compared to CMV promoter. (A) Luciferase activities of 3-copy synthetic promoters. (B) Luciferase activities of 6-copy synthetic promoters which have more than 8% of CMV promoter activity. Each synthetic promoters-luciferase constructs was transfected into H4IIE cells and luciferase activity was measured 1 day following transfection. Luciferase activities of synthetic promoter constructs were calculated as percentage of CMV promoter activity. LPK indicates pLPK-Luc plasmids. The luciferase assay was performed in quadruplicate in at least two different rounds of transfections. All values are means ±S.D.

FIG. 4 shows the composition of cis-regulatory elements in synthetic promoter constructs. The arrangement of cis-elements in the context of (A) 6-copy SPXI-Luc plasmids that showed over 8% of CMV promoter activity and (B) 9-copy SP-Luc plasmids that showed over 25% of CMV promoter activity. Over 300 clones of 6-copy SPXN-Luc and 6-copy SPXE-Luc were investigated for their transcriptional activity. Among them, 17 plasmids that showed their activity over 8% of that of CMV promoter were selected for the generation of 9-copy SP-Luc plasmids by inserting 3 additional copies of cis-elements. The selected 9-copy synthetic promoters were used for the production of adenovirus to determine their in vivo effects. The compositions of 6-copy SPXN are not shown.

FIG. 5 shows distribution patterns of transcriptional activities of (A) 6-copy SPXE-Luc, (B) 6-copy SPXN-Luc, and (C) 9-copy SPXN-Luc. Each DNA from 6-copy SPXE, 6-copy SPXN and 9-copy SP Luc was transfected into H4IIE cells, and then a luciferase assay was performed. Luciferase activities of synthetic promoter constructs were calculated by % of CMV promoter activity. The number of the constructs which have the indicated % of luciferase activity are shown.

FIG. 6 shows the effect of rAd-CMV-rINSfur on lowering blood glucose levels in STZ-induced diabetic NOD.scid mice. rAd-CMV-rINSfur viruses were administered into diabetic animals at different doses (5×10¹⁰, 10×10¹⁰, 5×10⁹, and 10×10⁹ viral particles). Representative data from each dosage group are shown. # indicates the death of treated animal on that day. •, 5×10¹⁰ viral particles; ∘, 10¹⁰ viral particles; ▾, 5×10⁹ viral particles; Δ, 10⁹ viral particles.

FIG. 7 shows the effect of rAd-23142-rINSfur on lowering blood glucose levels in STZ-induced diabetic NOD.scid mice. rAd-23142-rINSfur viruses were administered into diabetic mice at different doses of (A) 5×10¹⁰ (n=4), (B) 10×10¹⁰ (n=7), and (C) 5×10⁹ (n=6) viral particles. All value are means ±S.D.

FIG. 8 shows the effect of rAd-23137-rINSfur on lowering blood glucose levels in STZ-induced diabetic NOD.scid mice. (A) The effect of rAd-23137-rINSfur on lowering blood glucose level in diabetic animals. rAd-23137-rfNSfur and rAD-CMV-rINSfur viruses were administered into STZ-induced diabetic NOD.scid mice, and blood glucose levels were monitored. As a negative control, diabetic NOD.scid mice that were not treated with virus were used. •, rAd-23137-rINSfur (n=7); ∘, Diabetic NOD.scid mice (n=4); ▾, rAd-CMV-rINSfur (n=8). All values are means ±S.D. (B) Presence of recombinant adenoviral genome in the liver of treated animals. The livers were obtained from the mice treated with rAd-CMV-rINSfur (5×10⁹) or rAd-23137-rINSfur viruses (10¹⁰ viral particles) at different time following virus administration. Whole DNA was extracted from the livers of PCR and was performed to detect the adenoviral genome using primers for rat insulin gene. Lane 1, rAd-CMV-rINSfur on day 5; Lane 2, rAd-23137-rINSfur on day 10; Lane 3, rAd-23137-rINSfur on day 25; Lane 4, rAd-23137-rINSfur on Day 50; Lane 5, pAd-23137-rINSfur plasmids as positive control.

FIG. 9 shows a glucose tolerance test in rAd-23137-rINSfur-treated NOD.scid mice. Normoglycemic animals after treatment with rAd-23137-rINSfur were fasted for 16 hr, and glucose (2 g per kg body weight) was administered by intraperitoneal injection. Blood glucose levels were measured at 1, 15, 30, 60, 90, 120, 180, 240, and 270 min following glucose challenge. •, rAd-23137-rINSfur-treated NOD.scid mice; ∘, Normal control mice; ▾, Diabetic control mice. All values are means ±S.D.

FIG. 10 shows liver cell-specific insulin gene expression in rAd-23137-rINSfur-treated cell lines. rAd-23137-rINSfur or rAd-CMV-rINSfur viruses were infected into several cell lines as follows: (A) H4IIE (rat hepatoma cell line), (b) L929 (mouse fibroblast cell line), (C) L6 (mouse muscle cell line), (C) HeLa (Human cervical carcinoma cell line), (D) NRK (Normal rat kidney cell line), (E) 3T3-L1 (mouse fibroblast cell line). Cells were stained using specific antibody against rat insulin. Cells without virus infection were used as negative control.

FIG. 11 shows a liver-specific insulin gene expression in rAd-23137-rINSfur-treated NOD.scid mice. rAd-CMV-rINSfur and rAd-23137-rINsfur viruses were administered into STZ-induced diabetic NOD.scid mice. After the treated animals showed normal glucose levels, they were sacrificed and the various organs including kidney (K), spleen (S), liver (L), lung (Lu), and heart (H) were removed. Total RNAs were isolated and RT-PCR was performed using primers for rat insulin or HPRT. (A) rAd-CMV-rINSfur, HPRT, (B) rAd-CMV-rINSfur, rat insulin, (C) rAd-23137-rINSfur, HPRT, and (D) rAd-23137-rINSfur, rat insulin.

FIG. 12 shows the effect of rAd-23137-rINSfur on lowering blood glucose levels in spontaneous diabetic NOD mice. rAd-23137-rINSfur viruses (3×10¹⁰ viral particles, n=4) were administered into diabetic NOD mice intravenously via tail vein. The animals treated with rAd-23137-rINSfur showed normalized blood glucose levels a few days after administration, and normalized blood glucose levels were maintained for up to 10 days. All values are means ±S.D.

### DETAILED DESCRIPTION OF THE INVENTION

This invention is directed to the construction and use of tissue specific, glucose responsive promoters for the regulated expression of insulin in vivo. The synthetic promoters of the invention exhibited tightly regulated liver-specific activity of insulin expression in vivo in response to glucose. These functional attributes indicate that the regulated promoters of the invention can be used therapeutically for the treatment or cure of diabetes in humans through insulin gene therapy. In addition, the glucose responsive promoters of the invention exhibit several advantages that can are beneficial for the in vivo regulation of insulin expression. For example, the transcriptional activities of the synthetic promoters of the invention were relatively higher than natural glucose-responsive promoters such that blood glucose levels were maintained within normal ranges in vivo. The synthetic promoters also induced liver-specific expression of insulin gene, which can help ameliorate unexpected side effects due to the ubiquitous expression of insulin during in vivo gene therapy. Additionally, the synthetic promoters of the invention also exhibited more efficient glucose responsive abilities to clear exogenously introduced glucose quickly and decline insulin expression rapidly compared to natural glucose-responsive promoters.

In one embodiment, synthetic promoters composed of 3 distinctive transcription factor binding elements in random combinations were generated. Liver-enriched transcription factor binding elements were used for the synthetic promoter construction. Hepatocyte nuclear factor-1 (HNF-1) and the CAAT/enhancer binding protein (C/EBP) response elements were used for enhanced liver-specific transcriptional activity. The glucose responsive element (GRE) was used to confer glucose responsiveness. Individual elements were synthesized and assembled to generate 3-copy modules that contained 3 copies of each cis-element in all possible combinations. The synthetic promoters consisting of three 3-copy modules were cloned in reporter plasmids to identify synthetic promoters with high transcriptional activity in vitro. The resultant promoters derived liver-specific insulin gene expression in response to glucose change and sufficiently maintained the blood glucose level within normal ranges in both immunodeficient STZ-induced NOD.scid mice and immunocompetent diabetic NOD mice in non-fasting states.

In a further embodiment, targeting in vivo insulin expression to the liver has advantages for insulin gene therapy because it is a major target organ of insulin action, and plays an important role in glucose homeostasis (Taylor, R. & Agius, L. (1988). The biochemistry of diabetes. Biochem. J, 250:625-640). Moreover, the liver possesses the essential glucose-sensing components such as the glucose transporter (Rencurel, F., Waeber, G., Antoine, B., Rocchiccioli, F., Maulard, P., Girard, J. et al. (1996). Requirement of glucose metabolism for regulation of glucose transporter type 2 (GLUT2) gene expression in liver. Biochem. J, 314 (Pt 3), 903-909) and glucokinase (Bonini, C., Ferrari, G., Verzeletti, S., Servida, P., Zappone, E., Ruggieri, L. et al. (1 997). HSV-TK gene transfer into donor lymphocytes for control of allogeneic graft-versus-leukemia. Science 276:1719-1724) (Rencurel, F., Waeber, G., Antoine, B., Rocchiccioli, F., Maulard, P., Girard, J. et al. (1996). Requirement of glucose metabolism for regulation of glucose transporter type 2 (GLUT2) gene expression in liver. Biochem. J, 314 (Pt 3), 903-909). Thus, like the pancreatic β-cells, the liver has the intrinsic ability to respond to changes in blood glucose concentrations. From this point of view, the liver is a suitable target organ for insulin gene therapy for type 1 diabetes.

As used herein, the term “diabetes” is intended to mean the diabetic condition known as diabetes mellitus. Diabetes mellitus is a chronic disease characterized by relative or absolute deficiency of insulin which results in glucose intolerance. The term is intended to include all types of diabetes mellitus, including, for example, type I, type II, and genetic diabetes. Type I diabetes is also referred to as insulin dependent diabetes mellitus (IDDM) and also includes, for example, juvenile-onset diabetes mellitus. Type I diabetes is primarily due to the destruction of pancreatic β-cells. Type II diabetes mellitus is also known as non-insulin dependent diabetes mellitus (NIDDM) and is characterized, in part, by impaired insulin releast following a meal. Insulin resistance can also be a factor leading to the occurrence of type II diabetes mellitus. Genetic diabetes is due to mutations which interfere with the function and regulation of β-cells.

Diabetes is characterized as a fasting level of blood glucose greater than or equal to about 140 mg/dl or as a plasma glucose level greater than or equal to about 200 mg/dl as assessed at about 2 hours following the oral administration of a glucose load of about 75 g. The term “diabetes” is also intended to include those individuals with hyperglycemia, including chronic hyperglycemia and impaired glucose tolerance. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes mellitus.

As used herein, the term “treating” is intended to mean an amelioration of a clinical symptom indicative of diabetes. Amelioration of a clinical symptom includes, for example, a decrease in blood glucose levels or an increase in the rate of glucose clearance from the blood in the treated individual compared to pretreatment levels or to an individual with diabetes. The term “treating” also includes an induction of a euglycemic response in the individual suffering from disregulated hyperglycemia. Euglycemia refers to the range of blood glucose levels clinically established as normal, or as above the range of hypoglycemia but below the range of hyperglycemia. Therefore, a euglycemic response refers to the stimulation of glucose uptake to reduce the plasma glucose concentration to normal levels. For most adults, this level corresponds to the range in concentration of about 60-105 mg/dL of blood glucose and preferably between about 70-100 mg/dL, but can vary between individuals depending on, for example, the sex, age, weight, diet and overall health of the individual. Effective treatment of a diabetic individual, for example, would be a reduction in that individual's hyperglycemia, or elevated blood glucose levels, to normalized or euglycemic levels, with this reduction directly resulting from secretion of insulin. Alternatively, effective treatment would be a reduction in fasting blood glucose to levels less than or equal to about 140 mg/dL.

The term “treating” is also intended to include the reduction in severity of a pathological condition or a chronic complication which is associated with diabetes. Such pathological conditions or chronic complications are listed in Table 1 and include, for example, muscle wasting, ketoacidosis, glycosuria, polyuria, polydipsia, diabetic microangiopathy or small vessel disease, atherosclerotic vascular disease or large vessel disease, neuropathy and cataracts.

TABLE 1
Pathological Conditions Associated with Diabetes

Kidney

Glomerular microangiopathy

Diffuse glomerulosclerosis
Nodular glomerulosclerosis (Kimmel-stiel-Wilson disease)

Urinary infections

Acute pyelonephritis

Renal Failure

Necrotizing papillitis
Emphysematous pyelonephritis

Glycogen nephrosis (Armanni-Ebstein lesion)

Eye

Retinopathy

Nonproliferative retinopathy: capillary Microaneurysms,
retinal edema exudates, and hemorrhages
Proliferative retinopathy: proliferation of small vessels

Visual Failure

hemorrhage fibrosis, retinal detachment

Cataracts
Transient refractive errors due to osmotic changes in lens
Glaucoma due to proliferation of vessels in the iris
Infections

Nervous System

Cerebrovascular atherosclerotic disease: strokes, death
Peripheral neuropathy; peripheral sensory and motor cranial,
autonomic

Skin

Infections: folliculitis leading to carbuncles
Necrobiosis lipoidica diabeticorum: due to microangiopathy
Xanthomas: secondary to hyperlipidemia

Cardiovascular system

Coronary atherosclerosis: myocardial infarction, death
Peripheral atherosclerosis: limb ischemia, gangrene

Reproductive system

Increased fetal death rate (placental disease, neonatal
respiratory distress syndrome, infection)

General

Increased susceptibility to infection
Delayed wound healing

Additional complications also include, for example, a general increased susceptibility to infection and wound healing. The term “treating” is also intended to include an increase in the average life expectancy of a diabetic individual compared to a non-treated individual. Other pathological conditions, chronic complications or phenotypic manifestations of the disease are known to those skilled in the art and can similarly be used as a measure of treating diabetes so long as there is a reduction in the severity of the condition, complication or manifestation associated with the disease.

As used herein, the term “preventing” is intended to mean a forestalling of a clinical symptom indicative of diabetes. Such forestalling includes, for example, the maintenance of normal levels of blood glucose in an individual at risk of developing diabetes prior to the development of overt symptoms of the disease or prior to diagnosis of the disease. Therefore, the term “preventing” includes the prophylactic treatment of individuals to guard them from the occurrence of diabetes. Preventing diabetes in an individual is also intended to include inhibiting or arresting the development of the disease. Inhibiting or arresting the development of the disease includes, for example, inhibiting or arresting the occurrence of abnormal glucose metabolism such as the failure to transfer glucose from the plasma into the cells. Therefore, effective prevention of diabetes would include maintenance of glucose homeostasis due to glucose-regulated insulin expression in an individual predisposed to a diabetic condition, for example, an obese individual or an individual with a family history of diabetes. Inhibiting or arresting the development of the disease also includes, for example, inhibiting or arresting the progression of one or more pathological conditions or chronic complications associated with diabetes. Examples of such pathological conditions associated with diabetes are listed in Table 1.

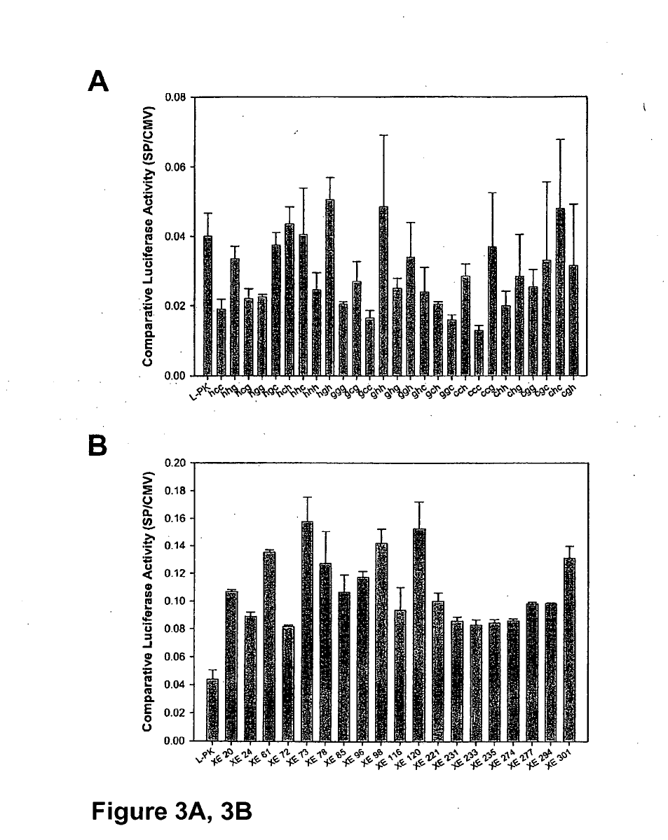

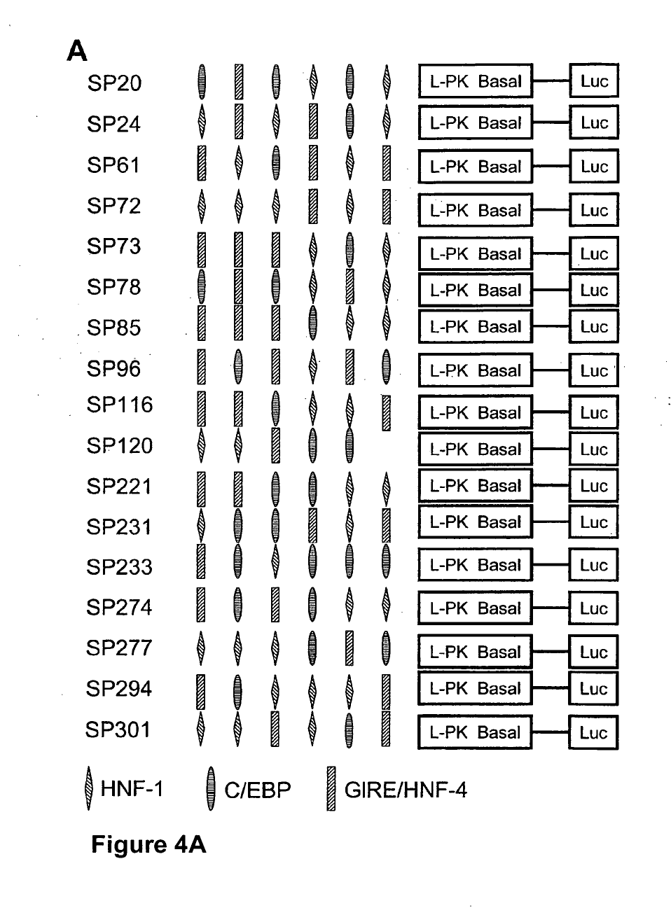

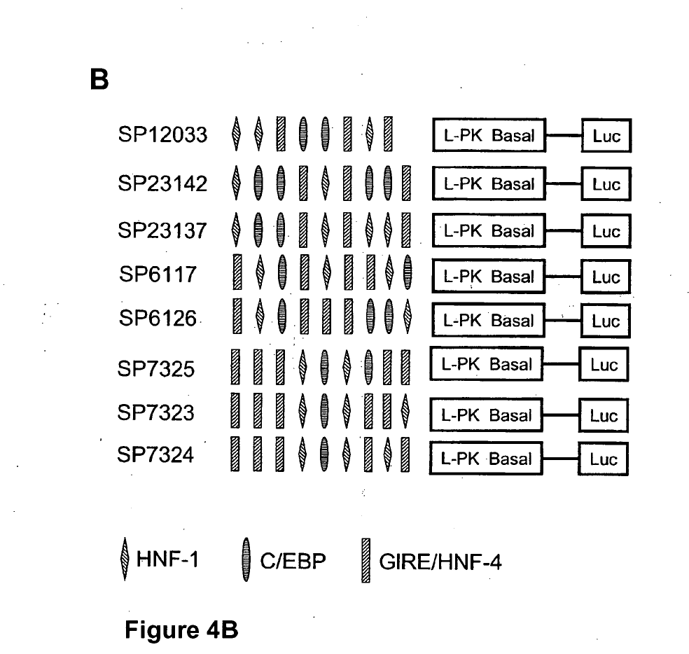

As used herein, the term “glucose responsive” is intended to mean the regulation of expression of a nucleic acid sequence by changes in levels of glucose. For example, glucose responsive expression includes the induction of promoter activity by increased levels of glucose. Such glucose responsive promoters include a tripartite transcription factor binding cis element such as a HNF-1, a C/EBP binding element and a GRE. Such glucose responsive promoters also include more than one tripartite transcription factor binding cis-element including 2, 3 or 4 or more tripartite transcription factor binding cis-elements. Specific examples of glucose responsive promoters containing one or more tripartite transcription factor binding cis elements are shown in, for example, FIGS. 3 and 4. A glucose responsive promoter also can contain a GRE without tissue specific or enhancer elements. Specific examples of glucose responsive elements found in other promoters include, for example, the rat ACC (-122) having the sequence CATGTGAAAACGTCGTG (SEQ ID NO:11); rat S₁₄ (-1439) having the sequence CACGTGGTGGCCCTGTG (SEQ ID NO:12); mouse S₁₄(-1439) having the sequence CACGCTGGAGTCAGCCC (SEQ ID NO:13); rat L-PK (-166) having the sequence CACGGGGCACTCCCGTG (SEQ ID NO:14), and rat FAS (-7210) having the sequence CATGTGCCACAGGCGTG (SEQ ID NO:15).

The term “glucose” when used in reference to the regulated expression of a gene is intended to include both glucose and glucose metabolites so long as such metabolites can cause increased expression from a glucose responsive promoter. A glucose metabolite includes those intermediate products of glucose metabolism such as glucose-6-phosphate, fructose-6-phosphate, glyceraldehyde-3-phosphate, glycerate-2-phosphate and pyruvate.

As used herein, the term “substantially” or “substantially the same” when used in reference to the proximity of one or more transcription factor binding cis elements is intended to mean that the elements are sufficiently close to allow interaction of a bound transcription factor with another trans binding factor, including a polymerase. The distances between transcription factor cis elements that are sufficient to carry out a function of a trans binding factor are well known in the art and can be found described in, for example, Bolouri and Davidson, Proc. Natl. Acad. Sci. USA 100:9371-9376 (2003) and Davidson et al., Science, 295:1669-78 (2002).

As used herein, the term “operationally linked,” or grammatical equivalents, is intended to mean that the nucleic acid components are joined according to well known genetic and cellular principles which allow the requisite function of each component to be carried out on its target nucleic acid. Therefore, an operationally linked group of cis elements that form tripartite a transcription factor binding cis element are joined together to cause transcription and regulation of a referenced coding region sequence. Similarly, an operationally linked insulin encoding nucleic acid coding region is joined to its promoter and tripartite transcription factor binding cis element such that the promoter will cause transcription and regulation of the insulin encoding nucleic acid.

As used herein, the term “expression” or grammatical equivalents are intended to mean the transcription, translation and processing of a nucleic acid by a cell. Expression can be, for example, constitutive or regulated such as by an inducible promoter or a tissue or cell specific promoter. Two or more nucleic acid sequences also can be expressed simultaneously or, alternatively, independently with other desired nucleic acids. A specific example of expressing two or more nucleic acid sequences includes the coexpression of insulin A and B chains. Various combinations of coexpression modes additionally can be used depending on the number and function of amino acid or nucleotide sequences being expressed. Those skilled in the art know, or can determine, what modes of coexpression can be used to achieve a particular goal or satisfy a desired need. Specific examples of both tissue specific and inducible expression using tripartite transcription factor binding cis elements are described further below in Example I.

As used herein, the term “insulin” is used to mean a polypeptide capable of stimulating glucose uptake by cells in response to increased glucose levels. Insulin can correspond to the amino acid sequence or any portion thereof from a variety of vertebrate species such as human, porcine, equine, rat or bovine so long as the resulting expressed molecule retains at least one bioactive function such as the stimulation of glucose uptake, glycogen synthesis, amino acid uptake, or protein synthesis. The term also includes modified forms of insulin having amino acid substitutions that enhance or do not greatly diminish the bioactivity of the polypeptide to stimulate glucose uptake by cells. Insulin also can include additions or deletions of amino acid residues so long as it retains insulin bioactivity. A bioactive insulin can therefore have an activity that is similar to wild type insulin or is higher or lower than wild type insulin so long as the bioactive insulin stimulates glucose uptake by cells.

Generally, human insulin has the molecular weight of about 5.8 kDa. The human insulin A- and B-chain sequences are provided as exemplary sequences for the insulin polypeptides of the invention. The A-chain of human insulin corresponds to nucleotides 2496-2591 (SEQ ID NO:7) and has the amino acid sequence show as SEQ ID NO:8, and the B-chain of human insulin corresponds to nucleotides 3477-3542 (SEQ ID NO:9) and has the amino acid sequence shown as SEQ ID NO:10. The human insulin gene sequence is deposited under the GenBank accession number J00265. The human insulin amino acid sequence is deposited under the GenBank accession number AAA59172. The human insulin cDNA sequence can be found at GenBank accession No. X70508. Nucleotide and amino acid sequences of insulin polypeptides from species other than human are known to those skilled in the art. All of these sequences as well as substantial equivalents and functional fragments thereof that maintain insulin bioactivity are included within the term “insulin” as used herein. In general, insulin consists of an A-chain region disulfide linked to a B-chain region.

Insulin A and B chains can derive from a proinsulin, which is a precursor form of insulin. Proinsulin polypeptides of the invention can be the amino acid sequence, or portions thereof, corresponding to a variety of vertebrate species such as human, porcine, equine, rat or bovine so long as it contains an A- or B-chain region of insulin, or a functional fragment thereof. Proinsulins of the invention include modified forms of proinsulin so long as the precursor polypeptide can be processed, or modified to be processed, into a bioactive form of insulin or into an A-chain or a B-chain region of insulin which is capable of assembling into a bioactive form of insulin. The precursor regions of the proinsulin polypeptides can be essentially any amino acid sequence so long as it does not negatively affect the processing of proinsulin into a bioactive form of insulin, an A-chain, a B-chain, or a functional fragment thereof. Therefore, the precursor region sequences can be, for example, the propeptide of insulin such as the proinsulin sequence intervening between a A-chain and a B-chain sequence. Alternatively, such precursor regions can be, for example, any of a variety of amino acid sequences, such as linker sequences, that are not normally found in vertebrate proinsulin molecules. The nucleotide and amino acid sequences of human insulin sequences are provided above as exemplary sequences for the proinsulin polypeptides of the invention . Nucleotide and amino acid sequences of proinsulin polypeptides from species other than human are known to those skilled in the art. All of these sequences as well as substantial equivalents and functional fragments thereof, that maintain their ability to be processed into bioactive insulin or into an A-chain or a B-chain region of insulin which is capable of assembling into a bioactive form of insulin are included within the term as used herein. Proinsulin can consist, for example, of a C-chain connected to the B- and A-chain sequences.

As used herein, the term “host cell” refers to a cell to be transformed or transduced by a nucleic acid, vector or viral particle of the invention. The term also refers to a cell that is capable of being infected by a viral particle containing a tissue specific glucose responsive promoter of the invention as its genome.

As used herein, the term “effective amount” when used in reference to the administration of viral particle having a vector expressing insulin in a tissue specific glucose responsive manner, is intended to mean that the number of administered viral particles is sufficient to infect a target tissue and express insulin from the viral particle's genome upon glucose induction at a level which will reduce one or more symptoms of diabetes. For example, an effective amount of viral particles expressing insulin consists of the number of particles that would cause a reduction in blood glucose levels or result in glucose homeostasis or both. Moreover, clinical manifestations of diabetes also can be used as a measure of an effective amount of viral particles as described above in Table 1. Similarly, an effective amount of viral particles also is intended to mean the number of viral particles that can be administered and direct the glucose regulated expression of insulin at sufficient levels to produce a desired effect on a biological or biochemical component cells or tissues of the individual. An effective amount of viral particles expressing insulin will result in normoglycemia upon food intake. An effective amount of viral particles for a human individual can be, for example, extrapolated from a credible animal model of diabetes given the teachings and guidance provided herein together with that well known by one skilled in the art. An effective of viral particles for a mouse animal model, for example, includes between about 1×10⁸-1×10¹², preferably between about 1×10⁹-1×10¹¹, more preferably between about 1×10¹⁰-1×10¹¹. A particularly useful effective amount is about 3×10¹⁰ viral particles.

As used herein, the term “pharmaceutically acceptable carrier” is intended to mean a solution or media which is appropriate for administration to an individual. Such solutions or media can act to maintain the stability of compounds and polypeptides and the viability of the cells. Pharmaceutically acceptable carriers are well known in the art and include aqueous solutions such as phosphate-buffered saline or media. A pharmaceutically acceptable carrier also includes additional moieties, compounds and/or formulations that act to enhance or increase the ability of the viral particles to target, attach or infect to their in vivo host cells or tissues and/or for timed released delivery or immunoprotection purposes. Such moieties, compounds and/or formulations are well known to those skilled in the art and can include, for example, receptor ligands, extracellular matrix molecules or components thereof and chemical delivery formulations.

As used herein, the term “isolated” is intended to mean a nucleic acid which is substantially free of contaminants or material as it is normally found in nature. Therefore, the term “isolated” includes a substantially pure nucleic acid as well as a nucleic acid introduced into a heterologous cell or tissue.

The synthetic promoters of the invention were designed and produced for the treatment of diabetes. Given the teachings and guidance provided herein it should be understood that the approach described herein is applicable to the design and treatment of a wide range of human pathologies. Such pathologies include gene replacement therapies as well as therapies that can regulate physiological systems through the introduction of one or more gene products. Accordingly, the invention is described with reference to gene replacement therapy of insulin for the treatment of diabetes but those skilled in the art will understand that the generation of synthetic promoters and regulated expression of a gene can be accomplished using the compositions and methods of the invention for a wide variety of diseases other than diabetes.

Briefly, one set of criteria for insulin gene replacement therapy includes selection of an appropriate target cell that has biochemical characteristics similar to β cells but autoimmune attack. The target cells also should contain the requisite biochemical machinery processing of proinsulin into mature, active insulin. A regulatory system which can express and release insulin in response to glucose levels also is beneficial to maintain glucose homeostasis.

With respect to the target cells, the liver can be used as an appropriate insulin-producing surrogate organ because it is a major location of insulin action, playing an important role in glucose homeostasis (Nguyen, T. H. & Ferry, N. (2004). Liver gene therapy: advances and hurdles. Gene Ther. 11 Suppl:S76-84. S76-S84). In addition, hepatocytes in the liver have a glucose sensing system similar to that found in pancreatic β cells, involving the glucose transporter 2 (Rencurel, F., Waeber, G., Antoine, B., Rocchiccioli, F., Maulard, P., Girard, J. et al. (1996). Requirement of glucose metabolism for regulation of glucose transporter type 2 (GLUT2) gene expression in liver. Biochem. J, 314 (Pt 3), 903-909) and glucokinase (Gould, G. W. & Holman, G. D. (1993). The glucose transporter family: structure function and tissue-specific expression. Biochem. J. 295 (Pt 2), 329-341; Iynedjian, P. B. (1993). Mammalian glucokinase and its gene. Biochem. J. 293 (Pt 1), 1-13). Thus, like the pancreatic β cells, the liver has the intrinsic ability to respond to changes in blood glucose concentrations.

Hepatocytes also contain a well characterized furin endoprotease with a well characterized cleavage recognition sequence (Arg-Xaa-Lys/Arg-Arg (Van, d., V, Roebroek, A. J., & Van Duijnhoven, H. L. (1993). Structure and function of eukaryotic proprotein processing enzmes of the subtilisin family of serine proteases. Crit Rev. Oncog., 4:115-136.). The cleavage recognition sequence can be incorporated into proinsulin as a heterologous substitute for its native cleavage sites to achieve processing into insulin A- and B-chains. Incorporation of a furin cleavage site can include changing the basic amino acid residue at the B/C (Arg-Arg) and C/A (Lys-Arg) junctions to a tetrabasic sequence (Arg-X-Lys-Arg; SEQ ID NO:16) which is recognized by furin. Alternatively, the insulin A- and B-chains can be co-expressed as mature, secretable polypeptides and allowed to self assemble into heterodimeric insulin.

Additionally, it also is beneficial to confer normal β cell regulation onto the insulin gene so that insulin will be produced and released in response to a rise in blood glucose levels, and suppressed as blood glucose concentrations decrease. Glucose regulation involves several factors in addition to transcriptional regulation and secretion including the need for a glucose transporter function (Rencurel, F., Waeber, G., Antoine, B., Rocchiccioli, F., Maulard, P., Girard, J. et al. (1996). Requirement of glucose metabolism for regulation of glucose transporter type 2 (GLUT2) gene expression in liver. Biochem. J, 314 (Pt 3), 903-909), glucokinase function and potassium/calcium channels which are utilized for the secretion of insulin in response to changes in extracellular glucose concentrations. Several attempts have been made to restore glucose-responsive biosynthesis of insulin in the liver using liver-specific and glucose-responsive promoters. However, transcriptionally regulated systems have relatively slow kinetics in both up- and down-regulation of insulin biosynthesis and therefore exhibit prolong the exposure to hyperglycaemia or lead to hypoglycaemia, respectively. The invention provides non-naturally occurring promoters that tightly regulate insulin production in response to glucose to circumvent these slow kinetics.

The invention provides an isolated tissue specific glucose responsive promoter. The promoter includes a polymerase binding domain 3′ to at lease one tripartite transcription factor binding cis element having a hepatocyte nuclear factor-1 (HNF-1) element, a CAAT/enhancer binding protein (C/EBP) response element and a glucose-response element (GRE).

The tissue specific glucose responsive promoters of the invention will have a polymerase binding site or a domain sufficient for recognition by a polymerase and initiation of transcription. The polymerase binding domain can constitute, for example, a type II polymerase binding domain. Such polymerases interact with the various transcription factors of the invention to augment or regulate mRNA transcription. However, other polymerase binding domains also can be utilized where corresponding modifications to the cis binding elements are made so as to be compatible with the chosen polymerase binding domain. A specific example of a polymerase binding domain is the liver pyruvate kinase (LPK) promoter fragment from −96 nucleotides (nt) to +12 nt relative to the transcription start site described below in Example I. Given the teachings and guidance provided herein, those skilled in the art will know that other eukaryotic promoters that actively transcribe message in the liver similarly can be utilized in the promoters and methods of the invention.

The tissue specific glucose responsive promoters of the invention include at least one tripartite transcription factor binding cis element. The tripartite element contains includes one or more transcription factor cis binding elements that augment transcription. The cis binding elements were chosen based on enhancement of transcription in the liver as described further below. However, for application of this invention to tissues other than liver, those skilled in the art will know that the cis binding elements active in the liver can be substituted with cis binding elements active in another target tissue when such other target tissue is being utilized for gene replacement therapy. Such other transcription factor cis binding elements active or specific to non-liver tissues are well known in the art. Therefore, the description below is exemplary with respect to cis element active in the liver.

Liver-specific glucose-responsive synthetic promoters were produced and selected to include one or more transcription factor cis binding elements that augment, for example, transcription in the liver. The production and selection of a variety of exemplary promoters with transcription factor cis binding elements 3′ to the LPK promoter is described in Example I and shown in FIGS. 3 and 4.

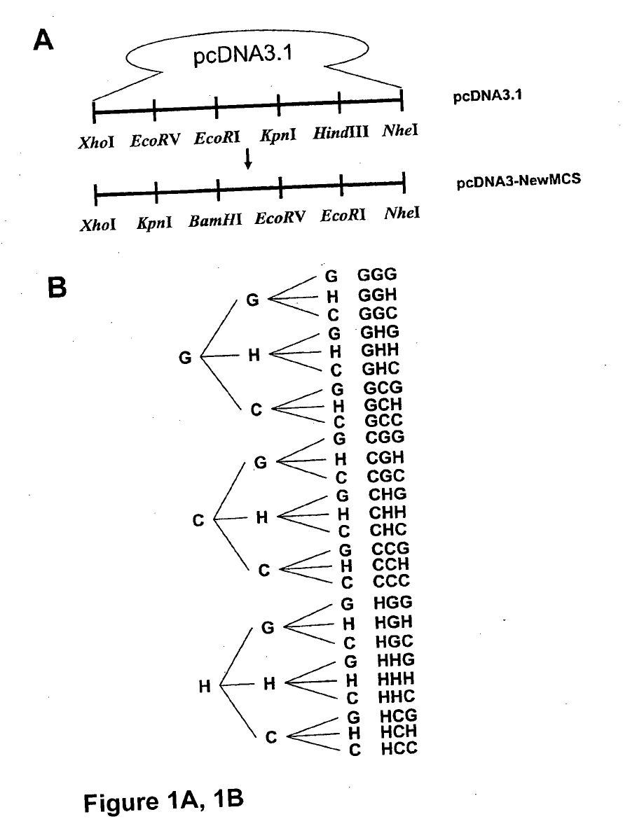

To achieve rapid transcriptional activation in a liver-specific manner, the promoters of the invention introduced hepatocyte-enriched transcription factor binding elements into a glucose-responsive system. Liver-specific promoters and enhancers as well the relative placement of cis-acting regulatory elements and their interactions with hepatic regulatory factors are well known in the art. Any known or identified transcription factor that augments transcription or control can be employed in the promoters of the invention. Exemplary liver specific transcription factor cis binding elements involved in positive regulation of liver specific gene expression include hepatocyte nuclear factor 1 (HNF-1), CAAT/enhancer binding protein (C/EBP), and hepatocyte nuclear factor 4 (HNF-4) (Cereghini, S. (1996). Liver-enriched transcription factors and hepatocyte differentiation. FASEB J, 10:267-282). Two or more of these factors function synergistically in distinctive combinations to stimulate cell-specific transcription and enhanced transcription of a given gene in adult hepatocytes appears to require a particular combination of these cis-elements. (De, S., V & Cortese, R. (1992). Transcription factors and liver-specific genes. Biochim. Biophys. Acta., 1132:119-126). For the specific example of a liver specific promoter of the invention, the tripartite sequence can include one or more of these factors to augment transcription of insulin in the liver. Synergistic combinations of two or more also can be included to the transcriptional activity and regulation of insulin. Synergistic combinations include, for example, those combinations shown in FIGS. 1-4 exhibiting high activity. Any of these combinations can be included in a tripartite transcription factor binding cis element within a promoter of the invention.

Glucose responsiveness of a tissue specific glucose responsive promoter of the invention can be achieved by, for example, including a glucose/carbohydrate response element (G1RE or ChoRE). Transcription factor cis binding sites other than GIRE also can be employed as a glucose responsive element of the invention. The GIRE element can be found in several 3′ regulatory regions of genes responsive to glucose including, for example, LPK and Spot 14 (Doiron, B., Cuif, M. H., Kahn, A., & az-Guerra, M. J. (1994). Respective roles of glucose, fructose, and insulin in the regulation of the liver-specific pyruvate kinase gene promoter. J. Biol. Chem., 269:10213-10216; Shih, H. M., Liu, Z., & Towle, H. C. (1995). Two CACGTG motifs with proper spacing dictate the carbohydrate regulation of hepatic gene transcription. J. Biol. Chem. 270:21991-21997; Shih, H. M. & Towle, H. C. (1992). Definition of the carbohydrate response element of the rat S14 gene. Evidence for a common factor required for carbohydrate regulation of hepatic genes. J. Biol. Chem., 267:13222-13228) and contains two copies of a motif related to the consensus binding site, CCTGTG, for the c-myc family of transcription factors. This binding site is sufficient for glucose-responsive regulation. However, GIRE requires an accessory site, such as HNF-4 for LPK and an unknown transcription binding site for S14, to support the full response to glucose (Shih, H. M., Liu, Z., & Towle, H. C. (1995). Two CACGTG motifs with proper spacing dictate the carbohydrate regulation of hepatic gene transcription. J. Biol. Chem. 270:21991-21997). The tissue specific glucose responsive promoters of the invention combine with a HNF-4 binding element with the LPK promoter to produce a hybrid element, designated GRE, that achieves enhanced glucose responding activity.

The order of transcription factor binding cis elements was selected based on activity of the resultant tissue specific glucose responsive promoters. Briefly, the regulatory regions of promoters and enhancers consist of a combination of several cis-regulatory elements, and the composition and arrangement of the elements determine the characteristics of the regulatory region through combinatorial interaction between transcription regulators (Carey, M. (1998). The enhanceosome and transcriptional synergy. Cell, 92:5-8). Transcription efficiency and activity level can be augmented through combinatorial and synergistic interaction of multiple activators bound to the cognate cis element with many different combinations of transcription factors being capable of transcriptional synergy. One benefit from these interactions is that a limited number of activators can be arranged in numerous possible combinations, each of which can be biologically distinct (Struhl, K. (2001). Gene regulation. A paradigm for precision. Science 293:1054-1055).

On the basis of these characteristics, libraries were produced and screened for particularly useful tissue specific glucose responsive promoters that contained random orders of three cis-regulatory elements: HNF-1 and C/EBP for liver-specific enhanced transcriptional activity; and glucose responsive element combined with HNF-4 (GRE) for glucose responsiveness of synthetic promoter. The synthetic promoter libraries were screened for promoters exhibiting high transcriptional activities using a rat hepatoma cell line in culture, followed by confirmation in vivo using an animal model.

The HNF-1 transcription factor binding cis element used for the construction of the tissue specific glucose responsive promoter libraries of the invention was about 27 nt in length and is show as SEQ ID NO: 1. However, HNF-1 cis elements useful in the promoters of the invention can include larger as well as smaller fragments so long as HNF-1 transcription activity is maintained. For example, an HNF-1 transcription factor binding cis element can range from between about 15-100 nucleotides, preferably about 20-50 nucleotides, and more preferably about 25-30 nucleotides.

The C/EBP transcription factor binding cis element used for the construction of the tissue specific glucose responsive promoter libraries of the invention was about 22 nt in length and is show as SEQ ID NO:2. However, C/EBP cis elements useful in the promoters of the invention can include larger as well as smaller fragments so long as C/EBP transcription activity is maintained. For example, aC/EBP transcription factor binding cis element can range from between about 12-100 nucleotides, preferably about 16-50 nucleotides, and more preferably about 20-25 nucleotides.

The GRE transcription factor binding cis element used for the construction of the tissue specific glucose responsive promoter libraries of the invention was about 48 nt in length and is show as SEQ ID NO:3. However, GRE cis elements useful in the promoters of the invention can include larger as well as smaller fragments so long as HNF-1 transcription activity is maintained. For example, an GRE transcription factor binding cis element can range from between about 15-100 nucleotides, preferably about 25-75 nucleotides, and more preferably about 45-50 nucleotides.

The cis elements within a tripartite transcription factor binding cis element of the invention can be incorporated in a variety of different combinations as described further below in Example I. The combinations can include different orders of individual elements including all possible combinations and permutations as well as spatial variations between the individual cis elements and between the tripartite element and the promoter element. Spatial variations can include, for example, cis elements substantially adjacent to one another or directly contiguous. Additionally, transcription factors also can operate at variously spaced locations where there is sufficient flexibility in the intervening region so that the nucleic acid can loop out while two factors physically interact while their cognate cis binding regions are located as substantial distances from one another. Therefore, spacing can be adjusted to include a wide range of intervening sequences between the transcription factor binding cis elements of the invention. Other modifications that can be included in the promoters of the invention include, for example, the size of the tripartite transcription factor binding cis element as described above. Specific combinations and sequences of the promoters of the invention are exemplified further in Example I and in FIGS. 1-4.

The tissue specific glucose responsive promoters of the invention contain at least one tripartite transcription factor binding cis element. The tripartite cis element is exemplified below having a wide variety of combinations of HNF-1, C/EBP and GRE. Other elements described above and below also can be substituted or included in this tripartite element to achieve augmentation of tissue specific expression or glucose responsiveness. In addition, to various combinations and permutations of component cis elements of the tripartite transcription factor binding cis element, various combinations, permutations and higher order repeats of the tripartite transcription factor binding cis element can be included in the promoters of the invention to further augment their transcription and regulatory control. For example, two or more tripartite transcription factor binding cis element can be combined substantially adjacent, continuous or containing intervening sequence to create a promoter having six or more component cis elements. The two or more tripartite elements can be combined or adjoined in the same or head-to-foot orientation. Alternatively, the two or more tripartite elements can be combined in the opposite or head-to-head orientation. As exemplified further in the Examples, either orientation will achieve the transcriptional activity and control useful in the methods of the invention.

Exemplified in the Examples are single tripartite elements as well as combinations of two and three tripartite transcription factor binding cis elements joined substantially adjacent, having only a restriction recognition sequence between each tripartite element. The activity of these tissue specific glucose responsive promoters are described in the Examples and exemplified in several assays in FIGS. 1-12. In addition to single, double or triple tripartite elements, other higher order repeats can be included in the promoters of the invention. Addition of more than 3 tripartite cis elements can similarly be in the same, opposite or any combination of the same or opposite orientation with respect to one another. Therefore, the invention provides a tissue specific glucose responsive promoter having 1, 2, 3, 4 or 5 or more tripartite transcription factor binding cis elements. The constituent transcription factor cis elements of the tripartite transcription factor binding cis elements can include HNF-1, C/EBP or GRE. Other elements described above and below also can be substituted or included in this tripartite element to achieve augmentation of tissue specific expression or glucose responsiveness.

In addition to construction, screening and identification of a repertoire of active tissue specific glucose responsive promoters in cultured cells, also referred to herein as in vitro, the transcriptional activity regulatory control of the promoters of the invention also were confirmed in vivo. Briefly, an adenoviral vector was employed for delivery of a nucleic acid encoding a heterologous gene under the control of a promoter of the invention, which confirmed similar in vivo transcriptional and regulatory activity of the various tripartite containing promoters of the invention compared to in culture measurements. Therefore, the invention also provides a host cell and a host having a cell which contains a tissue specific glucose responsive promoter of the invention that is capable of expressing a target nucleic acid. As described further below, the target nucleic acid can be insulin, proinsulin, insulin A-chain, insulin B-chain or both insulin A- and B-chains.

In a further embodiment of the invention, a host cell or a population of cells expressing proinsulin that can be processed to bioactive insulin in a tissue specific glucose responsive manner is provided. The host cells of the invention can originate from essentially any tissue or organ. One particularly applicable cell type for therapeutic treatments is a liver cell expressing proinsulin under a liver specific glucose responsive promoter of the invention that can be processed to insulin. However, any cell type can be employed as a host cell of the invention and can be selected based on its intended use including, for example, therapeutic, diagnostic or research purposes. For host primary cells, a tissue should be selected that is easily accessible and contains cells that exhibit desirable growth and expression characteristics. Additional considerations when selecting a tissue source include choice of a tissue that contains cells that can be isolated, cultured and modified to express bioactive insulin. Examples of sources of tissues other than liver include pancreas, muscle, fat, intestine, neuroendocrine cells or skin tissue, as well as sources of hematopoietic origin. Therefore, cell types within these tissues that can be modified to express a target gene in a tissue specific and glucose regulated manner and can be isolated and employed for purposes including, for example, experimental studies, vector maintenance and passage and for cell therapy protocols. Such cell types include, for example, hepatocytes, β islet cells, muscle (smooth, skeletal or cardiac), fibroblast, liver, fat, hematopoietic, epithelial, endothelial, endocrine, exocrine, kidney, bladder, spleen, stem and germ cells. Particularly useful host cells are hepatocytes, including progenitor and stem cells capable of differentiating into hepatocytes. Other cell types are similarly known in the art that are capable of being modified to express glucose responsive insulin and can similarly be obtained or isolated from a tissue source as described above. Although human tissue sources are advantageous for therapeutic purposes, the species of origin of the cells can be devised from essentially any mammal, so long as the cells exhibit the characteristics that allow for expression processing and secretion of proinsulin into bioactive insulin.

The invention also provides a method of treating or preventing diabetes. The method includes administering to an individual an effective amount of a viral particle having a vector comprising a tissue specific glucose responsive promoter comprising a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE) operationally linked to an insulin encoding nucleic acid, wherein expression of the insulin encoding nucleic acid is tissue specific and glucose responsive.

In a further embodiment, any of the tissue specific glucose responsive promoters of the invention can be operationally linked to an insulin encoding nucleic acid such as proinsulin and used gene replacement therapy. As described further below, any of a variety of methods well known in the art can be employed to introduce the tissue specific glucose responsive insulin encoding nucleic acid into a target cell or tissue for in vivo production of insulin and therapeutic treatment of diabetes. A particularly useful method for targeting and incorporation of an insulin encoding nucleic acid under the control of a promoter of the invention is to incorporate the gene construct into a viral vector to generate viral particles. Administration of such viral particles containing a vector of the invention is able to express bioactive insulin in a tissue specific and glucose responsive manner following infection for the treatment and prevention of diabetes.

In this regard, and as exemplified further below in Example I, recombinant adenoviruses can be produced that express an insulin encoding nucleic acid such as proinsulin gene under the control of the tissue specific glucose responsive promoters described herein and the viruses can be injected into an individual for infection and incorporation of the encoding nucleic acid and promoter of the invention into a recipient host cell. Depending on the viral vector, expression can be, for example, episomal or via incorporation into a host cell's genome. The encoding insulin nucleic acid can be human and be modified to introduce a furin-recognition site for the processing of proinsulin into mature insulin in the liver.

Adenoviral vectors are particularly useful for gene replacement therapy because they are well characterized and easily adaptable for use in human gene therapy.

Therefore, the viral particle exemplified in the methods of the invention is a adenoviral vector particle. However, as described previously and further below with respect to the vector, given the teachings and guidance provided herein, those skilled in the art will understand that a wide range of viral particles can be employed for tissue specific glucose responsive insulin gene delivery, expression and secretion of bioactive insulin. Whether adenoviral, other DNA virus-based, retroviral or other, the viral particles harboring a vector containing a therapeutic gene under the control of a tissue specific glucose responsive promoter of the invention as its genome can be employed in the methods of the invention for the therapeutic treatment or prevention of diabetes. The-viral particles of the invention can be produced, for example, using any of a wide variety of methods well known in the art for packaging viral genomes. Such methods are exemplified below in Example I with respect to an adenoviral particle harboring a vector of the invention.

A diabetic individual lacking glucose homeostasis can be treated with the above-described viral particles by a variety of administration routes and methods. An individual suitable for treatment using the methods of the invention is selected using clinical criteria and prognostic indicators of diabetes that are well known in the art. Definite clinical diagnosis of at least one of the symptoms of diabetes or pathologies related to diabetes as described previously herein would warrant administration of the cells of the invention. A list of exemplary pathological symptoms is included in Table 1.

An individual at risk of developing diabetes as assessed by known prognostic indicators such as family history, fasting blood glucose levels, or decreased glucose tolerance also warrant administration of cells modified to express proinsulin and protease in a glucose-regulated manner. One skilled in the art would recognize or know how to diagnose an individual with diabetes or disregulated glucose uptake and, depending upon the degree or severity of the disease, can make the appropriate determination of when to administer the viral particles of the invention and can also select the most desirable mode of administration. For example, whereas a person with long-standing type 1 disease can require immediate administration of viral particles for infection and expression of an insulin encoding nucleic acid under the control of a tissue specific glucose responsive promoter, a person with long-standing type 2 disease could defer treatment until after there is an indication of a lack of effectiveness of other prescribed treatments.

Viral particles having a vector containing a tissue specific glucose responsive promoter of the invention operationally linked to an insulin encoding nucleic acid such as proinsulin can be administered to an individual that has been determined to require or benefit from treatment for diabetes for amelioration of their disease. The viral particles can be administered for amelioration of one or more signs or symptoms of diabetes. For example, a diabetic individual can be administered viral particles having a genome containing a tissue specific glucose responsive promoter of the invention operationally linked to an insulin encoding nucleic acid for glucose regulated expression, processing and secretion bioactive insulin following diagnosis of the disease. The viral particles will infect target tissues and cells and express bioactive insulin in a glucose regulated manner upon in vivo expression of its vector genome. Insulin production will lead to, for example, restoration of glucose homeostasis. An individual that has been effectively treated for diabetes will exhibit a reduction in severity of at least one of the symptoms indicative of the disease following production of the heterologously produced insulin. The reduction in severity of a symptom can be determined and would be apparent to one skilled in the art.

Individuals with less severe diabetes can also be administered a viral particle of the invention. Determination of a need for treatment in such individuals can be made by one skilled in the art. For example, a diabetic individual that does not respond or responds poorly to standard treatment methods can be treated by methods of the invention. A patient with type 2 disease who has tried unsuccessfully to maintain a long-term decrease in weight or to adhere to an exercise regimen, for example, can be treated for their insulin resistance by implantation of a population of cells of the invention.

The methods of the invention can also be used to improve the efficacy of other therapies for diabetes. The methods of the invention can be used in combination with pre-existing or other methods of treatment to improve the efficacy or ease of use of the other methods. For example, the insulin producing liver cells can be generated following administration of the viral particles of the invention in a patient receiving daily injections of insulin or a patient using an insulin pump. Administration and infection with the viral particles containing a tissue specific glucose responsive promoter of the invention operationally linked to an insulin encoding nucleic acid with subsequent regulated production of bioactive insulin can reduce the frequency of insulin injections in such a patient. A diabetic individual not receiving insulin therapy but receiving behavioral modification therapy, for example, diet and exercise to decrease weight, also can be administered the viral particles of the invention. Administration of the viral particles containing a tissue specific glucose responsive promoter of the invention operationally linked to an insulin encoding nucleic acid in such individuals, in combination with a weight reduction and exercise regimen, can decrease the likelihood of disease relapse or can ameliorate signs or symptoms of the disease. The viral particles of the invention containing a tissue specific glucose responsive promoter of the invention operationally linked to an insulin encoding nucleic acid also can be used to treat a diabetic individual having autoimmune responses against endogenous insulin secreting cells. Such diabetic individuals are often treated by immunotherapeutic intervention of the autoimmune response. These individuals can be additionally treated through the liver specific glucose responsive production of bioactive insulin to achieve greater therapeutic efficacy than would be achieved with immunotherapy alone.

The viral particles of the invention containing a tissue specific glucose responsive promoter of the invention operationally linked to an insulin encoding nucleic acid can be administered to the individual to produce an increase in glucose homeostasis. Integration of the viral particle genome allows prolonged glucose homeostasis due to the expression restoration of insulin function. An individual suffering from diabetes can be administered an effective amount of viral particles to reduce or prevent diabetes. Such an individual could have a fasting blood glucose level of about 140 mg/dl or greater.

An effective amount of viral particles suitable for infection consists of a size or particle number that is within a range that can be obtained or modified to containing a tissue specific glucose responsive promoter of the invention operationally linked to an insulin encoding nucleic acid and is sufficient to express quantities of glucose regulated bioactive insulin following infection of the virus into a target cell or tissue that is therapeutically beneficial in vivo, such as the liver. An effective amount of viral particles for a human individual can be, for example, extrapolated from a credible animal model of diabetes given the teachings and guidance provided herein together with that well known by one skilled in the art. An effective of viral particles for a mouse animal model, for example, includes between about 1×10⁸-1×10¹², preferably between about 1×10⁹-8×10¹¹, more preferably between about 1×10¹⁰-1×10¹¹. A particularly useful effective amount is about 3×10¹⁰ viral particles. Choice of virus particle number can depend on the source of the particles, condition of the recipient individual, and the level of insulin secretion required. One skilled in the art will know, using methods well known in the art, how to determine the appropriate number of viral particles that produce a therapeutic effect.

Administration of the viral particles of the invention for delivery of insulin encoding nucleic acids expressed under the control of a liver specific glucose responsive promoter of the invention can be accomplished by a variety of routes. In addition to intravenously injection (i.v.), an effective amount of viral particles also can be administered into an individual by, for example, injection intramuscularly, subcutaneously, intraperitoneally, or into a tissue or organ site. Viral particles used for administration are obtained and prepared by methods well known in the art and suspended in an appropriate physiological carrier. For example, the viral particles can be infused either directly through a catheter connected to a device containing the particles and the catheter inserted into a vein, or can be injected directly into a tissue. The viral particles are injected in a pharmaceutically acceptable carrier which is defined above and further discussed below. The viral particles also can be administered with other molecules which facilitate delivery, targeting and/or therapeutic efficacy. The viral particles can be administered in single or multiple administrations as necessary to achieve sufficient expression of therapeutic levels of glucose regulated bioactive insulin.

The individual treated with the viral particles can then be monitored for efficacy of the treatment by measurement of levels of insulin secretion following ingestion of a meal. This measurement can consist of radioimmunoassay or ELISA measurement of, for example, insulin blood levels. Alternatively, measurement of fasting blood glucose levels in the individual following administration of the viral particles can be used to determine efficacy of the treatment. A decreased rate of glucose disposal as determined by a glucose tolerance test also can be used to verify efficacy of the treatment. Additionally, the alleviation of at least one of the symptoms associated with diabetes can also be used to determine efficacy of the treatment. One skilled in the art would know the appropriate means of evaluating and diagnosing efficacy of the treatment.

The invention can also be used for the prevention of diabetes. For example, viral particles containing a tissue specific glucose responsive promoter of the invention operationally linked to an insulin encoding nucleic acid can be administered as a prophylactic to an individuals at risk of developing diabetes or suffering from hyperglycemia. The invention can also be used, for example, in individuals genetically predisposed to developing diabetes or in obese individuals at risk for developing insulin resistance or disregulated hyperglycemia. These individuals can receive an effective amount of viral particles containing a tissue specific glucose responsive promoter of the invention operationally linked to an insulin encoding nucleic acid for infection of target cells and subsequent production of glucose regulated insulin prior to or during the onset of clinically overt hyperglycemia. The latter case can be considered as preventing the disease but can also be considered as treating the disease because normal glucose homeostasis is obtained before chronic elevated blood glucose levels are indicated.

In addition to administering viral particles containing a tissue specific glucose responsive promoter of the invention operationally linked to an insulin encoding nucleic acid for infection and production of glucose regulated bioactive insulin in an individual, the vectors of the invention also can be directly administered to an individual for genetic modification, for example, for ex vivo and in vivo therapy.

The viral particles or vectors of the invention containing a tissue specific glucose responsive promoter of the invention operationally linked to an insulin encoding nucleic acid can be introduced directly into an individual or formulated as a pharmaceutical composition including a pharmaceutically acceptable carrier. Pharmaceutically acceptable carriers are well known in the art and include aqueous solutions such as water, physiologically buffered saline, or other solvents or vehicles such as glycols, glycerol, oils such as olive oil or injectable organic esters.

A pharmaceutically acceptable carrier can contain physiologically acceptable compounds that act for example, to stabilize or increase the infection of the viral particle, absorption of the vector nucleic acid sequence or both. One skilled in the art will know that the choice of a pharmaceutically acceptable carrier, including a physiologically acceptable compound, depends, for example, on the route of administration of the viral particles containing a tissue specific glucose responsive promoter of the invention operationally linked to an insulin encoding nucleic acid and on the particular characteristics of the viral particles, for example, whether the viral particles are based on DNA viruses or retroviruses.

The pharmaceutical composition also can be incorporated, if desired, into oil-in-water emulsions, microemulsions, micelles, mixed micelles, liposomes, microspheres or other polymer matrices (Gregoriadis, Liposome Technology, Vols. I to III, 2nd ed., CRC Press, Boca Raton, Fla. (1993); Fraley et al., Trends Biochem Sci., 6:77 (1981). Liposomes, for example, which consist of phospholipids or other lipids, are nontoxic, physiologically acceptable and metabolizable carriers that are relatively simple to make and administer. In addition, liposomes are particularly useful because they can encapsulate the vectors containing a tissue specific glucose responsive promoter of the invention operationally linked to an insulin encoding nucleic acid of the invention with high efficiency while not compromising the biological activity of the agent, preferentially and substantially bind to a target cell, and deliver the aqueous contents of the vesicle into the target cell with high efficiency (see Mannino et al., Biotechniques 6:682 (1988)).

Targeting of a liposome for delivery of a vector of the invention to an individual can be passive or active. Passive targeting, for example, uses the tendency of liposomes to accumulate in cells of the reticuloendothelial system (RES) and in an organ such as the liver, which contains sinusoidal capillaries. The vectors formulated as liposomes can be infused directly into the portal vein of the liver and will effectively modify liver cells to express insulin due to the concentration of RES cells in the liver and the sinusoidal nature of the circulatory system in the liver. Active targeting of liposomes containing a vector can be achieved by coupling a specific ligand to the liposome. Such ligands include a monoclonal antibody, a sugar, a glycolipid or a protein such as a ligand for a receptor expressed by the target cells. Either method of targeting can be selected depending on the type of cell or location of tissue to be modified for insulin expression.

Administration of a viral particles or vectors containing a tissue specific glucose responsive promoter of the invention operationally linked to an insulin encoding nucleic acid to an individual can be as a single treatment or as multiple treatments depending on the level of insulin production or on the number of cells to be modified. Methods for the delivery of nucleic acid sequences encoding for a polypeptide are known in the art as described, for example, by Feigner et al., U.S. Pat. No. 5,580,859, issued Dec.3, 1996. Multiple administrations also can be performed to increase the proportion of modified cells, to increase the number of copies per cell of an insulin encoding nucleic acid operationally linked to a tissue specific glucose responsive promoter of the invention , or to maintain the effective number of modified cells for a desired duration. Efficacy of the in vivo treatment is achieved if at least one of the symptoms of diabetes is alleviated or reduced. A reduction in severity of a symptom of diabetes in a treated individual can be determined as described previously by one skilled in the art.

Exemplary modes of gene replacement therapy for introduction and expression of an insulin encoding nucleic acid under the control of a tissue specific glucose responsive promoter are exemplified below. Such modes include the use of adenoviral vectors and the tissue specificity is exemplified as liver.

There are three general components to consider in designing and optimizing a gene replacement therapy for the treatment of a disease. These components include a vector to transfer the gene, a device and procedure for delivering the vector to the appropriate tissue or organ, and a therapeutic gene. Each component can be chosen or modified based on criteria well known in the art depending on the specific characteristics of the particular disease of interest.

A number of gene transfer vehicles have been developed, any of which can be used in conjunction with the tissue specific glucose responsive promoters of the invention to deliver a regulated therapeutic gene. Gene transfer vehicles can roughly be divided into viral and non-viral gene delivery system.

Viral delivery systems are based on replicating viruses that are able to deliver genetic information into the host cell. In general, genomes of replicating viruses contain coding regions and cis-acting regulatory elements. The coding sequences are responsible for production of the viral structural and regulatory proteins that are essential for replication of infectious viruses, whereas cis-acting sequences are required for packaging of viral genomes and integration into the host cells. The coding regions of the virus are replaced by a therapeutic gene, leaving the cis-acting sequences intact to generate a replication-defective viral vector. When the viral vector is introduced into producer cells providing the structural viral proteins in trans, replication-defective virus particles containing the genetic information of a therapeutic gene are produced in those cells.

Viral vectors currently available for gene therapy are based on different viruses. Vectors based on adeno-associated virus (AAV) and retroviruses have the ability to integrate their viral genome into the chromosomal DNA of the host cell, which will possibly achieve lifelong gene expression. Vectors based on adenovirus and herpes simplex virus type 1 (HSV-1) represent the non-integrating vectors. These vectors deliver their genomes into the nucleus of the target cell, where they remain episomal.

Retroviruses are a large family of enveloped RNA viruses found in all vertebrates and can be classified into oncoretroviruses, lentiviruses, and spumaviruses. They have two copies of linear, positive-sense, single-stranded RNA genomes of 7 to 11 kbp. Following entry into target cells, the RNA genome is converted into linear double-stranded DNA by the viral enzyme reverse transcriptase and integrated into the cell chromatin (Goff, S. P. (2004). Retrovirus restriction factors. Mol. Cell., 16:849-859.). All retroviral genomes have two long terminal repeat (LTR) sequences at their ends (Wilhelm, M. & Wilhelm, F. X. (2001). Reverse transcription of retroviruses and LTR retrotransposons. Cell Mol. Life Sci., 58:246-1262.). LTRs and neighboring sequences act in cis during viral gene expression, and packaging, reverse transcription and integration of the genome. The gag, pol and env genes flanked by the LTRs encode the structural core proteins, nucleic-acid polymerases/integrases and surface glycoprotein, respectively. Lentiviruses have two additional genes in their genome such as tat and rev, essential for expression of the genome, and a variable set of accessory genes (Roebuck, K. A. & Saifuddin, M. (1999). Regulation of HIV-1 transcription. Gene Expr. 8:67-84.).

Due to the location of most cis-acting sequences in the terminal region, it is routine to produce retroviruses as viral gene transfer vectors. Up to 8 kb of therapeutic gene can be inserted and expressed in place of the viral genes. For the packaging of retroviral vector, the structural proteins are provided in trans in a packaging cell line (Mann, R., Mulligan, R. C., & Baltimore, D. (1983). Construction of a retrovirus packaging mutant and its use to produce helper-free defective retrovirus. Cell, 33:153-159). Construction of a retrovirus packaging mutant and its use to produce helper-free defective retrovirus. Cell, 33:153-159.). To prevent homologous recombination, packaging cells expressing structural proteins from separate constructs have been developed (Danos, O. & Mulligan, R. C. (1988). Safe and efficient generation of recombinant retroviruses with amphotropic and ecotropic host ranges. Proc. Natl. Acad. Sci. U.S.A, 85:6460-6464.).

The viral envelope glycoprotein dictates the host range of retroviral particles through its interaction with receptors on target cells. The cellular tropism can be modified through the substitution of particular viral Env by another one from different virus in a process as pseudotyping (Naldini, L., Blomer, U., Gage, F. H., Trono, D., & Verma, I. M. (1996). Efficient transfer, integration, and sustained long-term expression of the trans gene in adult rat brains injected with a lentiviral vector. Proc. Natl. Acad. Sci. U.S.A., 93:11382-11388; Zufferey, R., Nagy, D., Mandel, R. J. , Naldini, L., & Trono, D. (1997). Multiply attenuated lentiviral vector achieves efficient gene delivery in vivo. Nat. Biotechnol., 15:871-875). Such an approach can expand the host-range of retroviral vectors by incorporating sequences from unrelated viruses. For example, retroviral vectors pseudotyped with the G glycoprotein of the vesicular stomatitis virus (VSV-G), can infect most cells and can be concentrated to titers exceeding 1×10¹⁰ transduction unit/ml (Bums, J. C., Friedmann, T., Driever, W. , Burrascano, M., & Yee, J. K. (1993). Vesicular stomatitis virus G glycoprotein pseudotyped retroviral vectors: concentration to very high titer and efficient gene transfer into mammalian and nonmammalian cells. Proc. Natl. Acad. Sci. U.S.A., 90:8033-8037).

The ability of retroviral vectors to integrate into the genome of target cells is a useful characteristic for gene therapy application. Disruption of the nuclear membrane is required for the pre-integration complex to integrate into the chromatin (Roe, T. , Reynolds, T. C., Yu, G., & Brown, P. O. (1993). Integration of murine leukemia virus DNA depends on mitosis. EMBO J, 12:2099-2108.), and productive transduction by retroviral vectors is dependent on target cells mitosis shortly after entry (Miller, D. G., Adam, M. A. , & Miller, A. D. (1990). Gene transfer by retrovirus vectors occurs only in cells that are actively replicating at the time of infection. Mol. Cell Biol., 10:4239-4242.). Therefore, retroviral vectors can be applied to proliferative targets such as lymphocytes and hematopoietic progenitor cells (Halene, S. & Kohn, D. B. (2000). Gene therapy using hematopoietic stem cells: Sisyphus approaches the crest. Hum. Gene Ther., 11: 1259-1267.).

There are over 50 different human adenoviral serotypes. Among them, current vectors are primarily derived from those known as serotype 2 and 5. Moreover, secondary vector delivery using a different serotype capsid has been demonstrated in animal models for re-administration of a vector (Morral, N., O'Neal, W., Rice, K., Leland, M., Kaplan, J., Piedra, P. A. et al. (1999). Administration of helper-dependent adenoviral vectors and sequential delivery of different vector serotype for long-term liver-directed gene transfer in baboons. Proc. Natl. Acad. Sci U.S.A., 96:12816-12821; Parks, R., Evelegh, C., & Graham, F. (1999). Use of helper-dependent adenoviral vectors of alternative serotypes permits repeat vector administration. Gene Ther., 6:1565-1573.). Alteration of the cellular tropism or immunological response have been directed to changes in the viral fiber proteins from different serotypes responsible for the primary virus-cellular receptor binding (Curiel, D. T. (1999). Strategies to adapt adenoviral vectors for targeted delivery. Ann. N.Y. Acad. Sci., 886:158-171.; Wickham, T. J. (2000). Targeting adenovirus. Gene Ther., 7:110-114.).

After cell entry, the viral particle contains proteins that allow for efficient endosomal lysis and escape, allowing the genome to enter into the nucleus (Kasamatsu, H. & Nakanishi, A. (1 998). How do animal DNA viruses get to the nucleus? Annu. Rev. Microbiol., 52:627-686.). One cell can produce as many as 10,000 viral particles, and purified concentrations of 1×10¹³ vector particles/ml can be routinely achieved (Vorburger, S. A. & Hunt, K. K. (2002). Adenoviral gene therapy. Oncologist., 7:46-59.). The E3 genes are known to play a role in immune surveillance; however, they are dispensable for the viral life cycle (Wold, W. S. & Gooding, L. R. (1991). Region E3 of adenovirus: a cassette of genes involved in host immunosurveillance and virus-cell interactions. Virology, 184:1-8.). The removal of this region allows additional room for larger foreign DNA inserts up to 8 kb.

Useful vectors contain deletions of the E1 and E2 and/or E4 genes (Lusky, M., Christ, M., Rittner, K., Dieterle, A., Dreyer, D., Mourot, B. et al. (1998). In vitro and in vivo biology of recombinant adenovirus vectors with E1, E1/E2A or E1/E4 deleted. J. Virol., 72:2022-2032; Armentano, D., Smith, M. P., Sookdeo, C. C. , Zabner, J., Perricone, M. A. , St. George, J. A. et al. (1999). E40RF3 requirement for achieving long-term transgene expression from the cytomegalovirus promoter in adenovirus vectors. J. Virol. 73:7031-7034; Gorziglia, M. I., Lapcevich, C., Roy, S., Kang, Q., Kadan, M., Wu, V. et al. (1999). Generation of an adenovirus vector lacking E1, e2a, E3, and all of E4 except open reading frame 3. J. Virol. 73:6048-6055; Christ, M., Louis, B., Stoeckel, F., Dieterle, A., Grave, L., Dreyer, D. et al. (2000). Modulation of the inflammatory properties and hepatotoxicity of recombinant adenovirus vectors by the viral E4 gene products. Hum. Gene Ther. 11:415-427). To decrease possible viral toxicity, a helper-dependent vector system is available in which the helper virus contains all the viral genes required for replication with a conditional defect in the packaging domain, preventing it from being packaged into a viral capsid (Morsy, M. A. & Caskey, C. T. (1999). Expanded-capacity adenoviral vectors--the helper-dependent vectors. Mol. Med. Today, 5:18-24.). The second vector contains only the viral inverted terminal repeats (ITRs), therapeutic gene sequences and the normal packaging recognition signal, which allows this genome to be selectively packaged and released from cells. Due to the absence of viral genome except in the packaging region, these vectors have reduced toxicity (Balague, C., Zhou, J., Dai, Y., Alemany, R., Josephs, S. F., Andreason, G. et al. (2000). Sustained high-level expression of full-length human factor VII and restoration of clotting activity in hemophilic mice using a minimal adenovirus vector. Blood, 95:820-828; Morral, N., Parks, R. J., Zhou, H., Langston, C., Schiedner, G., Quinones, J. et al. (1998). High doses of a helper-dependent adenoviral vector yield supraphysiological levels of alpha 1-antitrypsin with negligible toxicity. Hum. Gene Ther., 9:2709-2716). The vector DNA genome exists episomally and is not replicated in transduced cells. Therefore, transgene expression can be lost over time due to either the dilution in replicating cells or degradation of the episomal genome in non-dividing cells. Expression can be restored through additional administrations of the therapeutic vector.

Although most adenovirus vectors transduced mainly the liver through intravenous administration, direct injection of adenoviral vector can also transduce most tissues (Vrancken Peeters, M. J., Perkins, A. L., & Kay, M. A. (1996). Method for multiple portal vein infusions in mice: quantitation of adenovirus-mediated hepatic gene transfer. Biotechniques, 20:278-285). These vectors have been used in preclinical animal studies to transducer liver, skeletal muscle, heart, brain, lung, pancreas and tumors (Bramson, J. L., Graham, F. L., & Gauldie, J. (1995). The use of adenoviral vectors for gene therapy and gene transfer in vivo. Curr. Opin. Biotechnol. 6:590-595).

AAV is a member of the dependoviruses, a subfamily of the parvoviridae. The virus is non-pathogenic and by itself nonreplicating. Therefore, it requires a helper virus, such as adenovirus, to mediate a productive infection (Muzyczka, N. (1992). Use of adeno-associated virus as a general transduction vector for mammalian cells. Curr. Top. Microbiol. Immunol. 158:97-129). There are six known serotypes, each of which may have a different cellular tropism. Because of the non-pathogenic characteristic of AAV, it is considered as a particularly useful vehicle for gene therapy. The viral genome consists of two genes: rep and cap. The cap gene encodes for the structural proteins that form the viral capsid, whereas the regulatory proteins are produced from the rep gene (Muzyczka, N. (1992). Use of adeno-associated virus as a general transduction vector for mammalian cells. Curr. Top. Microbiol. Immnunol. 158:97-129; Russell, D. W. & Kay, M. A. (1999). Adeno-associated virus vectors and ematology. Blood, 94:-874.; Monahan, P. E. & Samulski, R. J (2000). AA V vectors: is clinical success on the horizon? Gene Ther., 7:24-30; Tal, J. (2000). Adeno-associated virus-based vectors in gene therapy. J. Biomed. Sci., 7:279-291). These two genes are flanked by viral ITRs that are 145 nucleotides in length. Each viral particle contains a single double-strand genome. The packaging capacity of AAV is about 5.0 kb, which is a major limitation of this vector system (Monahan, P. E. & Samulski , R. J. (2000). Adeno-associated virus vectors for gene therapy: more pros than cons? Mol. Med. Today, 6:433-440). The wild-type virus in the presence of rep has a propensity to integrate into a specific region of human chromosome 19. However, this property is lost in vectors due to the absence of the rep gene (McCarty, D. M., Young, S. M., Jr., & Samulski R. J. (2004). Integration of adeno-associated virus (AAV) and recombinant AAV vectors. Annu. Rev. Genet. 38:819-845).

AAV vectors can be produced by replacement of rep and cap genes by promoter and transgene sequence. During recombinant AAV production, cap and rep sequences are provided from helper plasmids, and infectious viruses are easily rescued by coinfection with adenovirus (Matsushita, T., Elliger, S., Elliger, C., Podsakoff, G., Vilarreal, L., Kurtzman, G. J. et al. (1998). Adeno-associated virus vectors can be efficiently produced without helper virus. Gene Ther., 5:938-945; Xiao, X., Li, J., & Samulski, R. J. (1998). Production of high-titer recombinant adeno-associated virus vectors in the absence of helper adenovirus. J.Virol., 72:2224-2232). Other vector production systems have been developed that are free of replicating adenovirus (Xiao, X., Li, J., & Samulski, R. J. (1998). Production of high-titer recombinant adeno-associated virus vectors in the absence of helper adenovirus. J.Virol., 72:2224-2232), reducing possible contamination of wild type adenoviruses.

AAV vectors have been shown to transduce cells both through episomal transgene expression and by random chromosomal integration (Nakai, H., Iwaki, Y., Kay, M. A., & Couto, L. B. (1999). Isolation of recombinant adeno-associated virus vector-cellular DNA junctions from mouse liver. J.Virol., 73:5438-5447; Miao, C. H., Snyder, R. O., Schowalter, D. B., Patijn , G.A., Donahue , B., Winther, B. et al. (1998). The kinetics of rAAV integration in the liver. Nat. Genet., 19:13-15). AAV vectors also allow splitting a gene or expression cassette into two vectors and simultaneously administering them to muscle or liver (Yan, Z., Zhang, Y., Duan, D., & Engelhardt, J. F. (2000). Trans-splicing vectors expand the utility of adeno-associated virus for gene therapy. Proc. Natl. Acad. Sci. U.S.A, 97:6716-6721; Sun, L., Li, J., & Xiao, X. (2000). Overcoming adeno-associated virus vector size limitation through viral DNA heterodimerization. Nat.Med. 6:599-602; Nakai, H., Storm, T. A., & Kay, M. A. (2000). Increasing the size of rAAV-mediated expression cassettes in vivo by intermolecular joining of two complementary vectors. Nat. Biotechnol., 18:527-532). Further, since the AAV vector genome lacks viral coding sequences, the vector itself has not been associated with toxicity or any inflammatory response except for the generation of dsDNA genomes by either vector ssDNA annealing, or second strand-synthesis followed by vector genome linking to form concatemers (Nakai, H., Storm, T. A., & Kay, M. A. (2000). Recruitment of single-stranded recombinant adeno-associated virus vector genomes and intermolecular recombination are responsible for stable transduction of liver in vivo. J.Virol., 74:9451-9463; Vincent-Lacaze, N., Snyder, R. O., Gluzman, R., Bohl, D., Lagarde, C., & Danos O. (1999). Structure of adeno-associated virus vector DNA following transduction of the skeletal muscle. J.Virol., 73:1949-1955; Duan, D., Yan, Z., Yue, Y., & Engelhardt, J. F. (1999). Structural analysis of adeno-associated virus transduction circular intermediates. Virology, 261:8-14; Yang, J., Zhou, W., Zhang, Y., Zidon, T., Ritchie, T., & Engelhardt, J. F. (1999). Concatamerization of adeno-associated virus circular genomes occurs through intermolecular recombination. J.Virol., 73:9468-9477; Ferrari, F. K., Samulski, T., Shenk, T., & Samulski, R. J. (1996). Second-strand synthesis is a rate-limiting step for efficient transduction by recombinant adeno-associated virus vectors. J.Virol., 70:3227-3234; Fisher, K. J. , Gao G. P., Weitzman, M. D., DeMatteo, R., Burda, J. F., & Wilson J.M. (1996). Transduction with recombinant adeno-associated virus for gene therapy is limited by leading-strand synthesis. J.Virol., 70:520-532). The vector particle can be delivered into many different organs, such as the central nervous system (CNS), liver lung and muscle by in vivo administration (Monahan, P. E. & Samulski , R. J. (2000). AA V vectors: is clinical success on the horizon? Gene Ther. 7:24-30), and AAV vectors have been found to efficiently transduce non-dividing cells (Miao, C. H. , Nakai, H;, Thompson, A. R., Storm, T. A., Chiu, W., Snyder, R. O. et al. (2000). Nonrandom transduction of recombinant adeno-associated virus vectors in mouse hepatocytes in vivo: cell cycling does not influence hepatocyte transduction. J.Virol. 74:3793-3803).

Herpes simplex virus type 1 (HSV-1) is an engineered herpes virus for purposes of gene transfer. The vector exhibits a large capacity for foreign gene insertion of at least 30 kb of non- HSV sequences allowing large single genes or multiple transgenes that can be coordinately or simultaneously expressed (Krisky, D. M., Marconi, P. C., Oligino, T. J., Rouse, R. J. , Fink, D. J., Cohen, J. B. et al. (1998). Development of herpes simplex virus replication-defective multigene vectors for combination gene therapy applications. Gene Ther. 5:1517-1530).

The HSV amplicon vectors system is based on the ability of HSV-1 to package defective genomes containing the cis-acting sequences, ori (origin of viral DNA replication) and pac (packaging and cleavage signal). HSV amplicon vectors contain no viral genes except cis-acting elements (Spaete, R. R. & Frenkel, N. (1982). The herpes simplex virus amplicon: a new eucaryotic defective-virus cloning-amplifying vector. Cell, 30:295-304). They are generally approximately 15 kb in length. The standard amplicon system requires the functions of helper HSV for particle production and packaging of genome-length concatemerized vector DNA. Amplicon vector production has been improved through the use of helper virus genome plasmids deleted for packaging signals; the helper genomes are propagated in bacteria as bacterial artificial chromosomes (Saeki, Y., Ichikawa, T., Saeki, A., Chiocca, E. A., Tobler, K., Ackermann, M. et al. (1998). Herpes simplex virus type 1 DNA amplified as bacterial artificial chromosome in Escherichia coli: rescue of replication-competent virus progeny and packaging of amplicon vectors. Hum. Gene Ther., 9:2787-2794). This packaging system markedly decreases the generation of replication competent virus and cytotoxicity.

Non-viral vectors exhibit certain advantages as well which include:: (1) they are easy to prepare and to scale-up, (2) they are more flexible with regard to the size of the DNA being transferred, (3) they are generally safe in vivo and (4) they minimize elicitation of a specific immune response, and can therefore be administered repeatedly. A number of studies have been reported using naked DNA, DNA-cationic-liposome complexes, DNA-polymer complexes and combinations of these (Lollo, C. P., Banaszczyk, M. G., & Chiou, H. C. (2000). Obstacles and advances in non-viral gene delivery. Current Opinion In Molecular Therapeutics, 2:136-142; Feigner, P. L. , Barenholz, Y., Behr, J. P. , Cheng, S. H., Cullis, P., Huang, L. et al. (1997). Nomenclature for synthetic gene delivery systems. Human Gene Therapy, 8:511-512; Zauner, W., Ogris, M., & Wagner, E. (1998). Polylysine-based transfection systems utilizing receptor-mediated delivery. Advanced Drug Delivery Reviews, 30:97-113; Ledley, F. D. (1995). Nonviral gene therapy: The promise of genes as pharmaceutical products. Human Gene Therapy, 6:1129-1144).

Various non-viral, pharmaceutical formulations of genes for human therapy also are available, particularly with ligand-directed targeting of DNA-cationic-liposome complexes (Lipoplexes). Lipoplexes combined with ligands such as folate, transferrin or anti-transferrin-receptor antibody have achieved targeted gene delivery and expression in human breast, prostate, head and neck cancers (Rolland, A. P. (1998). From genes to gene medicines: recent advances in nonviral gene delivery. Critical Reviews In Therapeutic Drug Carrier Systems, 15:143-198; Audouy, S. A. L., de Leij, L. F. M. H., Hoekstra, D., & Molema, G. (2002). In vivo characteristics of cationic liposomes as delivery vectors for gene therapy. Pharmaceutical Research, 19:1599-1605).

Synthetic chemical vectors also can be used as a vehicle for delivery due to their stability and the potential ease of chemical modification. In addition, the low cost consistent standard of production, greater safety and high flexibility also make these compounds particularly useful (Lollo, C. P., Banaszczyk, M. G., & Chiou, H. C. (2000). Obstacles and advances in non-viral gene delivery. Current Opinion In Molecular Therapeutics, 2:136-142). Furthermore, non-viral agents can be used in different combinations, providing flexibility in achieving particular therapeutic aims.

Using physical gene delivery methods, naked DNA is delivered directly to the cytoplasm, bypassing endosomes and lysosomes, and hence avoiding enzymatic degradation. Skin gene therapy, for example, is typically associated with direct delivery methods, including biolistic or microprojectile induction, topical application, direct injection and electroporation (Vogel, J. C. (2000). Nonviral skin gene therapy. Human Gene Therapy, 11:2253-2259).

One non-viral gene transfer system currently in use for gene therapy is the injection of naked plasmid DNA (pDNA) into local tissues or the systemic circulation (Horn, N. A., Meek, J. A. , Budahazi, G., & Marquet, M. (1995). Cancer gene therapy using plasmid DNA: Purification of DNA for human clinical trials. Human Gene Therapy, 6:565-573). For example, it has been reported that transgene constructs injected into muscle tissue or liver tissue in the form of naked-DNA molecules may be taken up and expressed by muscle and liver cells (Herweijer, H. & Wolff, J. A. (2003). Progress and prospects: naked DNA gene transfer and therapy. Gene Ther., 10:453-458). Naked-DNA gene transfer systems are composed of a plasmid that contains the cDNA of therapeutic gene under the transcriptional control of a various eukaryotic regulatory elements (Hartikka, J., Sawdey, M., Cornefert-Jensen, F., Margalith, M., Barnart, Nolasco, M. et al. (1996). An improved plasmid DNA expression vector for direct injection into skeletal muscle. Human Gene Therapy, 7:1205-1217; Lui, V. W., Falo, L. D., Jr., & Huang, L. Systemic production of IL-12 by naked DNA mediated gene transfer: toxicity and attenuation of transgene expression in vivo. The Journal Of Gene Medicine, 7:384-393).

Degradation of naked DNA can be circumvented using a large-volume injection (Christianson, S. W., Shultz, L. D., & Leiter, E. H. (1993). Adoptive transfer of diabetes into immunodeficient NOD-scid/scid mice. Relative contributions of CD4+ and CD8+ T-cells from diabetic versus prediabetic NOD.NON-Thy- 1a donors. Diabetes, 42:44-55) which induces efficient gene transfer in internal organs such as the liver. Physical pressure caused by a large volume of plasmid-containing solution delivers pDNA into cells directly, resulting in transgene expression in target cells (Schultz, J., Pavlovic, J., Strack, B., Nawrath, M., & Moelling, K. (1999). Long-lasting anti-metastatic efficiency of interleukin 12-encoding plasmid DNA. Human Gene Therapy, 10:407-417; Blezinger, P., Wang, J., Gondo, M., Quezada, A., Mehrens, D., French, M. et al. (1999). Systemic inhibition of tumor growth and tumor metastases by intramuscular administration of the endostatin gene. Nature Biotechnology, 17:343-348).

Electrotransfer also can be used as a mode for delivery of a vector or nucleic acid containing a tissue specific glucose responsive promoter of the invention operationally linked to an insulin encoding nucleic acid. Briefly, pDNA is injected intramuscularly in a high volume of a physiological solution, allowing distribution of DNA through the tissue by convection. Electric pulses are then applied, generally using external electrodes. The parameters of in vivo pDNA electrotransfer are known in the art and are applicable for use in the methods of the invention.

Further, jet injection of a solution containing DNA also can be used to transfer genes to skin, muscle, adipose and mammary tissue. Several jet injectors have been developed, and are commonly used to deliver corticosteroid and anaesthetic solutions into human skin using an air propulsion system.

Another gene delivery mode includes ultrasound. Therapeutic ultrasound induces cell membrane permeabilization for increasing the transfection efficiency of naked pDNA into skeletal muscle and other tissues. For example, the ultrasound contrast agent, Optison (Molecular Biosystems, San Diego, USA), contains gas-filled human albumin microspheres, which can be loaded with DNA by mixing them with plasmids. Optison can enhance the transfection efficiency of naked pDNA in vivo as well as in vitro (T Taniyama, Y., Tachibana, K., Hiraoka, K., Aoki, M., Yamamoto, S., Matsumoto K. et al. (2002). Development of safe and efficient novel nonviral gene transfer using ultrasound: Enhancement of transfection efficiency of naked plasmid DNA in skeletal muscle. Gene Therapy, 9:372-380). In addition, intra-uterine injection of naked DNA, in combination with microbubble-enhanced ultrasound, has been shown to produce high levels of protein expression in fetal mice (Endoh, M., Koibuchi, N., Sato, M., Morishita, R., Kanzaki, T., Murata, Y. et al. (2002). Fetal Gene Transfer by Intrauterine Injection with Microbubble-Enhanced Ultrasound. Molecular Therapy, 5:501-508). Ultrasound-contrast-agent microbubbles are capable of amplifying the targeting of genes to particular tissues through the combination of site-specific ligands. Furthermore it is possible to increase payload volume by co-injecting gene-bearing vehicles, such as liposomes, with the microbubbles (Price, R. J. & Kaul, S. (2002). Contrast ultrasound targeted drug and gene delivery: An update on a new therapeutic modality. Journal of Cardiovascular Pharmacology and Therapeutics, 7:171-180).

In vivo gene transfer using pDNA can employ delivery systems such as liposomes or cationic polymers to protect it from degradation, when administered systematically. Polycation addition leads to electrostatic neutralization of anionic charges of pDNA molecules, and condenses the polynucleotide structure, thereby protecting it against nuclease digestion. pDNA and cationic amphiphiles can be formulated in different ratios to produce complexes of diverse size and surface-charge properties. Additionally, complexes bearing a net positive charge display enhanced binding to negatively charged cell membranes, leading to increased cellular uptake (Rolland, A. P. (1998). From genes to gene medicines: recent advances in nonviral gene delivery. Critical Reviews In Therapeutic Drug Carrier Systems, 15:143-198).

Gene transfer using DNA-cationic-liposome complexes (lipoplexes) also has been used successfully as a method for delivering therapeutic genes. Cationic lipoplexes are routine easy and inexpensive to produce. They are made up of non-toxic and non-immunogenic materials can deliver large polynucleotides into somatic cells. In addition, these reagents are readily engineered in the laboratory to incorporate novel biological functions, or to produce new formulations that can be screened for in vivo activity. Furthermore, liposome-mediated gene transfer does not generate any antivirus immune response, and has less risk of generating tumourigenic mutations because the delivered gene has a low integration frequency and generally does not replicate in transfected cells. Stabilization of particles or liposomes generally increases biocompatibility, reduces immune response, increases in vivo stability and delays clearance from circulation.

Polymer-based systems using collagen, lactic or glycolic acid, or polyanhydride are additional modes for delivering therapeutic genes in vivo. First, pDNA molecules within the polymer can be protected against degradation in the circulation system until they are released into target cells. Second, injection or implantation of the polymer into the body can be easily manipulated to target a particular cell type or tissue. For example, targeting of gene transfer can be achieved by modification of gene carriers using cell targeting ligands, such as anti-CD3 and anti-CD5 antibodies for T cells, or transferrin for some cancer cells (Gijsens, A., Derycke, A., Missiaen, L., De Vos, D., Huwyler, J. , Eberle, A. et al. (2002). Targeting of the photocytotoxic compound AIPcS4 to Hela cells by transferring conjugated PEG-liposomes. International Journal of Cancer, 101:78-85; Hofland, H. E. J., Masson, C., Iginla, S., Osetinsky, J., Reddy, J. A., Leamon, C. P. et al. (2002). Folate-Targeted Gene Transfer in Vivo. Molecular Therapy, 5:739-744; Bohl, K. E., Bergstrand, N., Carlsson, J., Edwards, K., Johnsson, M., Sjoberg, S. et al. (2002). Development of EGF -conjugated liposomes for targeted delivery of boronated DNA-binding agents. Bioconjug. Chem., 13:737-743). In addition, cationic-liposome-based transfection complexes conjugated with folate have been shown to specifically transfect folate-receptor-expressing cells and tumors, suggesting that this is a potential therapy for intraperitoneal cancers (Reddy, J. A., Abburi, C., Hofland, H., Howard, S. J., Vlahov, I., Wils, P. et al. (2002). Folate-targeted, cationic liposome-mediated gene transfer into disseminated peritoneal tumors. Gene Therapy, 9:1542-1550).

Transductional targeting is one mode of delivery that can be used in vivo for therapeutic gene replacement therapy. For example, viral vectors can be used to specifically target a tissue or cell type or they can be used to broadly introduce a gene into a broad range of cell types depending on the virus used and its host cell specificity. In this regard, recombinant AAV vectors are beneficial in gene therapy applications because they allow transfer transgenes into a wide range of target cells. Increasing the efficiency and specificity of viral vectors to particular cell populations will enhance the safety of gene therapy by allowing lower viral loads to be administered. Modifying the vector capsid, which plays an important role in determining cellular tropism to achieve tissue targeting (transductional targeting) also is applicable to the methods of the invention. There are several methods to modify the cellular tropism of gene transfer vectors such as pseudotyping, conjugation of capsid with molecular adaptors with particular receptor-binding properties, and genetic targeting of tropism.

One form of transductional retargeting, which requires little prior knowledge of specific virus-receptor interactions, is pseudotyping. Pseudotyping involves replacement of capsid from a certain viral serotype with one from another viral serotype. Pseudotyping has been used to generate selective retroviruses as well as selective chimeric adenovirus vectors (Somia, N. & Verma, J. M. (2000). Gene therapy: trials and tribulations. Nat. Rev. Genet., 1:91-99.). In addition, AAV vector genomes flanked by AAV2 inverted terminal repeats have also been successfully cross-packaged in the capsids of different serotypes, creating a portfolio of pseudotypes with different specificities (Rabinowitz, J. E., Rolling, F., Li, C., Conrath, H., Xiao, W., Xiao, X. et al. (2002). Cross-packaging of a single adeno-associated virus (AAV) type 2 vector genome into multiple AAV serotypes enables transduction with broad specificity. J.Virol., 76:791-801.; Grimm, D. & Kay, M. A. (2003). From virus evolution to vector revolution: use of naturally occurring serotypes of adeno-associated virus (AAV) as novel vectors for human gene therapy. Curr. Gene Ther. 3:281-304; Gao, G. P., Alvira, M. R., Wang, L., Calcedo, R., Johnston, J., & Wilson, J. M. (2002). Novel adeno-associated viruses from rhesus monkeys as vectors for human gene therapy. Proc. Natl. Acad. Sci. US.A, 99:11854-11859).

Conjugation of the viral capsid with molecular adaptors having particular receptor-binding properties also can be used to deliver a vector containing a tissue specific glucose responsive promoter of the invention operationally linked to an insulin encoding nucleic acid. A second method for targeting vector capsids to distinct cell populations has been to conjugate capsids with molecular adaptors containing particular receptor-binding properties. This approach has been used to enhance the transduction of various cultured cell types using adenovirus (Douglas, J. T., Miller, C. R., Kim, M., Dmitriev, J., Mikheeva, G., Krasnykh, V. et al. (1999). A system for the propagation of adenoviral vectors with genetically modified receptor specificities. Nat. Biotechnol., 17:470-475), retrovirus (Snitkovsky, S. & Young, J. A. (2002). Targeting retroviral vector infection to cells that express heregulin receptors using a TVA-heregulin bridge protein. Virology, 292:150-155) and AAV vectors (Ponnazhagan, S., Mahendra, G., Kumar, S., Thompson, J. A. , & Castilas, M., Jr. (2002). Conjugate-based targeting of recombinant adeno-associated virus type 2 vectors by using avidin-linked ligands. J.Virol., 76:12900-12907).

A third approach to administer the promoters of the invention operationally linked to a insulin encoding nucleic acid is to genetically engineer the capsid genes to abolish normal receptor binding ability of recombinant vector or to incorporate a small peptide ligand for alternative receptor binding into the capsid structure. This genetic approach to transductional retargeting has been successful at redirecting adenovirus vector tropism in several studies (Hidaka, C., Milano, E., Leopold, P. L., Bergelson, J. M., Hackett, N. R., Finberg, R. W. et al. (1999). CAR-dependent and CAR-independent pathways of adenovirus vector-mediated gene transfer and expression in human fibroblasts. J.Clin. Invest, 103:579-587).

Transcriptional targeting is a further mode for selectively delivering a therapeutic gene. The tissue specific glucose responsive promoters of the invention exemplify the transcriptional targeting of expression to the liver.

The use of strong eukaryotic promoters such as those described herein can be used, for example, to achieve long-term expression in vivo compared to the use of strong viral promoters (Verma, M. (2003). Viral genes and methylation. Ann. N.Y. Acad. Sci., 983:170-180.). The use of cell-selective eukaryotic promoters provides the additional benefit of specific expression of transgene.

Guidance as to promoter structure and design considerations are described further below including, for example, core promoter structure, transcriptional factor, and rational design of promoters. This guidance is applicable to the design and use of the tissue specific glucose responsive promoters of the invention.

Briefly, eukaryotic gene expression is generally controlled through the activity of RNA polymerase II (RNAPII) and the basal transcription factors that are recruited to the core promoter elements found in every eukaryotic gene (Orphanides, G. & Reinberg, D. (2002). A unified theory of gene expression. Cell, 108:439-451). The RNAPII and its basal transcription factors such as TFIIB and TFIID are assembled upon several regulatory elements in the core promoter spanning the region −35 to +35 bp relative to transcriptional start site (Roeder, R. G. (1996). The role of general initiation factors in transcription by RNA polymerase II. Trends Biochem. Sci., 21:327-335). The regulatory elements contain the TATA box and TFIIB recognition element (BRE) in TATA box containing promoters whereas the initiator and downstream binding element (DBE) are located in TATA-less promoters.

The TATA box is generally located at −25 bp in many regulatory regions of eukaryotic gene, but notably is absent from the promoters of many housekeeping genes (Smale, S. T. (1997). Transcription initiation from TATA-less promoters within eukaryotic protein-coding genes. Biochim. Biophys. Acta., 1351:73-88). The initiator (Inr) is generally located on the transcription start site at −3 to +5 bp, and is able to support transcription in the absence of a TATA box, or synergizing with it when both are present (Martinez, E., Zhou, Q., L'Etoile, N. D., Oelgeschlager, T., Berk, A. J., & Roeder R. G. (1995). Core promoter-specific function of a mutant transcription factor TFIID defective in TATA-box binding. Proc. Natl. Acad. Sci. U.S.A., 92:11864-11868). The DPE is also found around +30 bp and appears to act as an alternative to the TATA box in TATA-less promoters. The BRE which plays a role in providing a bridge between promoter-bound TFIID and RNAPII, is located upstream of the TATA box (Littlefield, 0., Korkhin, Y., & Sigler, P. B. (1999). The structural basis for the oriented assembly of a TBP/TFB/promoter complex. Proc. Natl. Acad. Sci. U.S.A., 96:13668-13673).

The many basal transcription factors involved in transcription are used to recruit RNAPII to the promoter via these motifs, and hence to form the pre-initiation complex (PIC) to start transcription at the start site (Cornetta, K., Morgan, R. A., Gillio, A., Sturm, S., Baltrcki, L., O'Reily, R. et al. (1991). No retroviremia or pathology in long-term follow-up of monkeys exposed to a murine amphotropic retrovirus. Hum. Gene Ther., 2:215-219). The transcription factors in the PIC include cell type-specific components in the basal transcription machinery (Albright, S. R. & Tjian, R. (2000). TAFs revisited: more data reveal new twists and confirm old ideas. Gene, 242:1-13; Freiman, R. N., Albright, S. R., Zheng, S., Sha, W. C., Hammer, R. E., & Tjian, R. (2001). Requirement of tissue-selective TBP-associated factor TAFII105 in ovarian development. Science, 293:2084-2087). Additionally, tissue-specific basal components also can be used to further increases the tissue-specificity through combinatorial transcription factor/basal factor interaction (Holmes, M. C. & Tjian, R. (2000). Promoter-selective properties of the TBP-related factor TRF1. Science, 288:867-870; Rabenstein, M. D., Zhou, S., Lis, J. T., & Tjian, R. (1999). TATA box-binding protein (TBP)-related factor 2 (TRF2), a third member of the TBP family. Proc. Natl. Acad. Sci. U.S.A, 96:4791-4796; Yamit-Hezi, A. & Dikstein, R. (1998). TAFII105 mediates activation of antiapoptotic genes by NF-kappaB. EMBO J., 17:5161-5169).

Whereas the basal machinery is necessary to generate basal levels of transcription in vitro, its interaction with additional cis-acting regulatory factors provide a further level of control. These cis-elements are recognized by gene-, stimuli- or tissue-specific transcription factors that act as trans-activators or -repressors of gene expression, depending on the context.

Transcription factors control the activity of the basal machinery generally via coregulators, which act as mediators between basal factors and transcription factors (Glass, C. K. & Rosenfeld, M. G. (2000). The coregulator exchange in transcriptional functions of nuclear receptors. Genes Dev., 14:121-141; Lemon, B. & Tjian, R. (2000). Orchestrated response: a symphony of transcription factors for gene control. Genes Dev., 14:2551-2569). These coregulators play important roles in generating promoter- and tissue-specific gene expression and can be used to further increase the tissue specificity of the promoters of the invention. They are also the means by which cells are able to affect global networks of transcription, and orchestrate gene expression in response to various stimuli and at various stages of development (Lemon, B. & Tjian, R. (2000). Orchestrated response: a symphony of transcription factors for gene control. Genes Dev., 14:2551-2569). The short cis-elements that bind to these factors therefore offer a range of choices and designs of promoters for targeting and controlling heterologous transgene expression under a glucose responsive element and are particularly useful to modify expression to achieve a specific target or response. Transcription factor binding sites are located either up or downstream of the transcription start site. Transcription factors bound to these elements are brought into proximity with the promoter by looping of the intervening DNA during activation.

Rational design of promoters for transcriptional targeting also can be employed to generate a tissue specific glucose responsive promoter of the invention. In this regard, a comprehensive understanding of transcriptional mechanisms is not a prerequisite for the generation of de novo synthetic promoters. Functional screening of promoter constructs as described in Example I can be used to identify optimal combinations of different elements that contain the desired activity, which can be synthesized randomly in short lengths or be generated by random combination of different cis-elements into a composite promoter (Edelman, G. M., Meech, R., Owens, G. C., & Jones, F. S. (2000). Synthetic promoter elements obtained by nucleotide sequence variation and selection for activity. Proc. Natl. Acad. Sci. U.S.A., 97:3038-3043; Li, X., Eastman, E. M., Schwartz, R. J., & Draghia-Akli, R. (1999). Synthetic muscle promoters: activities exceeding natually occurrng regulatory sequences. Nat. Biotechnol., 17:241-245.).

It is understood that modifications which do not substantially affect the activity of the various embodiments of this invention are also included within the definition of the invention provided herein. Accordingly, the following examples are intended to illustrate but not limit the present invention.

### EXAMPLE I

### Insulin Gene Therapy for the Treatment of Type 1 Diabetes

This Example shows the generation and selection of multipartite glucose responsive promoters and their use for delivering regulated insulin levels to diabetic animals.

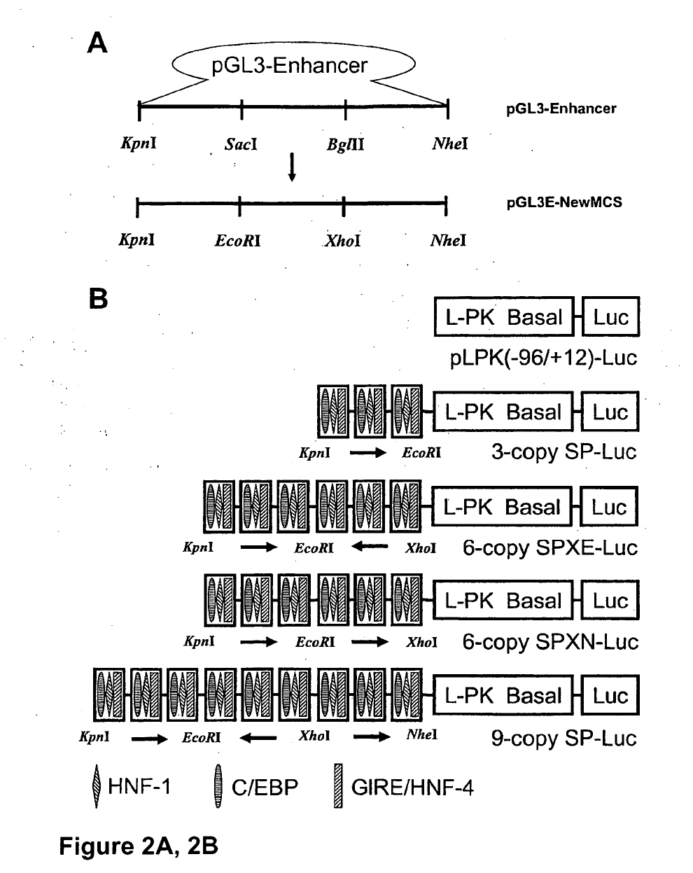

Synthetic promoter libraries were constructed by first generating a 3-copy module of cis elements. The module contained 3 copies of the transcription factor binding cis elements for the hepatocyte nuclear factor-1 (HNF-1), CAAT/enhancer binding protein (C/EBP) response element and the glucose-response element (GRE) in all possible combinations. As described below, the combinations were generated by sequential insertion of each cis-element in 3 restriction enzyme sites as shown in FIG. I B. These 3-copy modules were transferred to pLPK(−96/+12)-Luc plasmids to generate 3-copy SP-Luc as shown in FIG. 2. In addition, 6-copy SP-Luc plasmids were generated by insertion of 3-copy modules into 3-copy SP-Luc plasmids.

Briefly, for construction of the promoter-harboring plasmids the original multiple cloning sites (MCS) of the pGL3-Enhancer plasmid (Promega Madison, Wis., USA) was replaced with a new MCS containing XhoI, KpnI, BamHI, EcoRV, EcoRI and NheI sites in sequence, generating pGL3E-NewMCS plasmid (FIG. 1A). pLPK-TA plasmid was constructed using direct insertion of PCR amplified LPK promoter region (−3200 bp/+12 bp relative to transcriptional start site) into the TA cloning vector (Invitrogen, Burlington, ON, Canada). A 2.5 kbp SacI/HindIII-digested fragment of LPK promoter was removed from pLPK-TA plasmid, and then cloned into the same sites of pGL3-Enhancer, generating pLPK-luc plasmid that was used as a positive control in the promoter assay. A 108 bp NheI/HindIII-digested fragment of LPK promoter (−96 bp/+12 bp relative to transcriptional start site) was removed from the pLPK-TA, and was inserted into the same sites of pGL3E-NewMCS plasmid to generate PLPK(−96/+12)-luc plasmid which was used as the basal promoter of all synthetic promoters generated in the present study and is shown in FIG. 1B. pCMV -Luc plasmid was generated by transferring luciferase gene at HindIII/XbaI site ofpGE3-Enhancer to the same site of pcDNA3.1/Hygro plasmid that was purchased from Invitrogen (Burlington, ON, Canada).

To produce random arrangement of individual cis-elements, all 27 different plasmids of 3-copy SP-Luc were mixed at the amount of 10 μg. In addition, the 3-copy modules excised from 3-copy SP-D3 plasmid were mixed together and ligated with the same enzyme-cut mixture of 3-copy SP-Luc, generating 6-copy SP-Luc. To cover the number of possible combination of 6 cis-elements (3), we isolated more than 300 clones from both 6-copy SPXE- and SPXN-Luc plasmids.

For the generation of a synthetic promoter library, two complementary oligonucleotides were synthesized for each control element phosphorylated, and annealed to yield short DNA fragments. The oligonucleotides sequences were as follows: HNF-1 (sense), 5′-CTAGCTGGTTAATGATTAACCAGGACT-3′ (SEQ ID NO:1); HNF-1 (antisense), 5′-CTAGCTGGTTAATGATTAACCAGGACT-3′ (SEQ ID NO:4); C/EBP (sense), 5′-TCGCAAGTTGCGCAATATCGCG-3′ (SEQ ID NO:2); C/EBP (antisense), 5′-TCGCAAGTTGCGCAATATCGCG -3′ (SEQ ID NO:5); Glucose responsive elements (sense), 5′-GGGCGCACGGGGCACTCCCGTGGTTCCTGGACTCTGGCCCCCAGTGTA-3′ (SEQ ID NO:3), Glucose responsive elements (antisense), 5′-TACACTGGGGGCCAGAGTCCAGGAACCACGGGAGTGCCCCGTGCGCCC-3′ (SEQ ID NO:6). All synthetic fragments were synthesized with specific restriction enzyme cohesive ends at both ends of oligonucleotide for the generation of the synthetic promoter library. The introduced restriction enzyme sites were as follows: KpnI/BamHI, BamHI/EcoRV and EcoRV/EcoRI.

Two complementary oligonucleotides (100 pmol each) were mixed in 20 ul of annealing solution (10 mM Tris-HCl pH 7.9, 2 mM MgCl2, 50 mM NaCl, and 1 mM EDTA), and incubated at 90° C. for 5 min and allowed to cool slowly to room temperature. The annealed oligonucleotide fragments were phosphorlyated using T4 polynucleotide kinase (NEB, Beverly, Mass., USA) in 50 ul of reaction mixture (70 mM Tris-HCl pH7.6, 10 mM MgCl2, 5 mM DTT) for 30 min at 37° C., followed by heat inactivation at 70° C. for 10 min. The annealed/phosphorlyated DNA fragments were purified by phenol/chlrolform/isoamyl alcohol extraction, and then used for the generation of synthetic promoter libraries.

First, each three cis-element containing KpnI/BamHI cohesive end were cloned into pcDNA3-NewMCS plasmid that was digested with the same enzyme, generating 1-copy SP-D3 plasmids, where another three cis-element fragments were cloned into BamHI/EcoRV and EcoRV/EcoRI sites in sequence to generate 3-copy SP-D3 plasmids (FIG. 1). To generate synthetic promoter-reporter plasmids, the KpnI/EcoRI-digested DNA fragments (3-copy modules) from 3-copy SP-D3 plasmid were subcloned into the same sites of pGL3E-NewMCS, generating 3-copy SP-Luc plasmids (FIG. 2B).

For generation of 6-copy SP-Luc plasmid, 10 ug of each 3-copy SP-D3 were mixed and digested with either XhoI/EcoRI or XhoIUNheI followed by gel extraction of 3-copy modules. Concurrently, all 3-copy SP-Luc plasmids were mixed in the same way and digested with same enzyme sets. The eluted 3-copy modules were subcloned into the pool of XhoI/EcoRl- or XhoI/NheI-digested 3-copy SP-Luc, generating 6-copy synthetic promoter libraries in the same direction (6-copy SPXN-Luc) or opposite direction (6-copy XPXE-Luc) with previously inserted 3-copy module (FIG. 2B). The transformants that contain 6-copy SPXN-Luc or 6-copy SPXE-Luc plasmids were cultured and harvest for the plasmid preparation using miniprep kit (Qiagen, Mississauga, ON, Canada). After screening for high transcriptional activity of synthetic promoters, additional 3-copy synthetic promoters fragments from 3-copy SP-D3 were inserted into the selected 6-copy SPXE-Luc at the sites of XhoI/Nhel sites to generate 9-copy SP-Luc plasmids.

The screening of synthetic promoter libraries was performed as described below. Initially, the large number of clones to be screened for high transcriptional activity imposed a practical challenge for assessing a representative number of different promoter combinations. To solve this problem, plasmids of low quality instead of plasmids of high quality that required time- and labour-consuming purification steps were used. Although low quality plasmids showed about 10% transfection efficiency of high quality plasmids the promoter activities of both low and high quality plasmids were found to be similar, indicating that the results from low quality plasmids is representative of those from high quality plasmids. Thereafter, all transfection experiments were used with low quality plasmids.

All plasmids used in the transfection experiment for initial screening of synthetic promoter were prepared using miniprep kit from Qiagen according to the manufacturer's instruction. Midiprep kit (Qiagen, Mississauga, ON, Canada) was used to isolate plasmids for the comparison of transfection efficiencies with low quality plasmids from miniprep kit. H4IIE cells were cultured as described below. One day before transfection, cells were seeded into 96-well plates at a number of 5000 cells/well. Cells were transfected with 150 ng plasmid/well using lipofectamine plus reagent (Invitrogen, Burlington, ON, Canada) according to the manufacturer's instruction and collected 24 h post-transfection. Cells were lysed with Glo Lysis Buffer (Promega, Madison, Wis., USA), and luciferase activity was measured using Steady-Glo Luciferase assay system (Promega, Madison, Wis., USA). The assay was performed in quadruplicate in at least two different rounds of transfection.

The transcriptional activities of 3-copy SP-Luc plasmids were examined as the first step of synthetic promoter screening using rat hepatoma cell line. The results are shown in FIG. 3A. As a control, CMV promoter which drives a strong ubiquitous expression of the target gene was used. In addition, LPK promoter, a naturally occurring liver-specific glucose-responsive promoter, was used as a control for naturally occurring promoter. Most of the 3-copy SP-Luc plasmids had activities between 2 to 5% of CMV promoter activity and comparable to that of LPK promoter. Promoters consisting of only multimerized single elements had activities lower than LPK promoter (FIG. 3A), which is consistent with previous report (Li, X., Eastman, E. M., Schwartz, R. J., & Draghia-Akli, R. (1999). Synthetic muscle promoters: activities exceeding natually occurrng regulatory sequences. Nat. Biotechnol., 17:241-245).

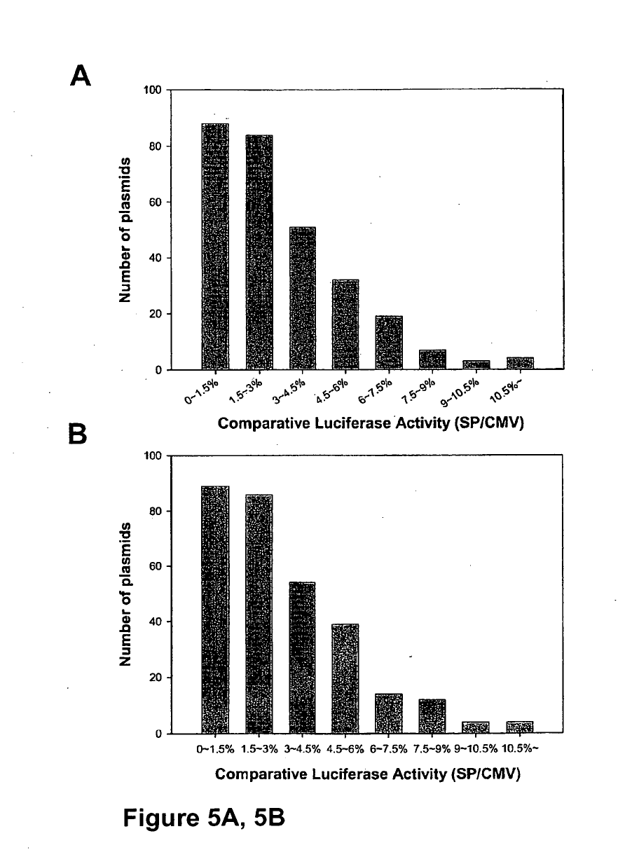

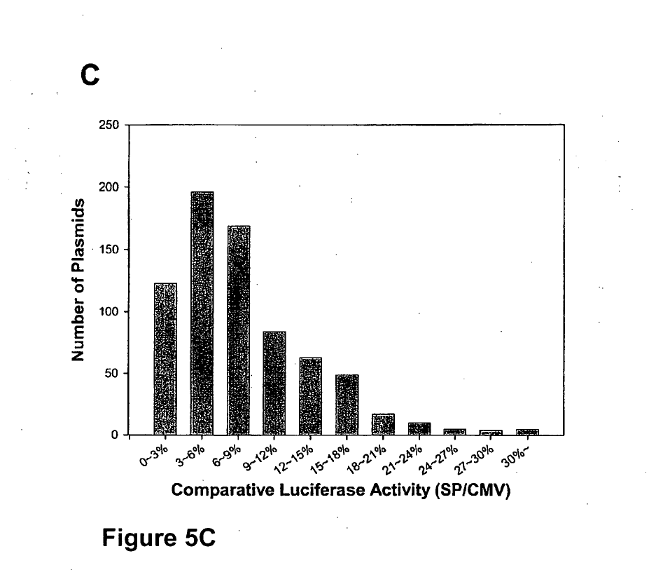

An additional 3 copy module was incorporated into 3-copy SP-Luc in the same or opposite direction with a previously constructed 3-copy module in order to examine the transcriptional activities in 6-copy SP-Luc and the effect of element orientation (FIG. 2B). More than 300 clones from XE and XN were examined for their transcriptional activities. No significant differences were found in distribution of transcriptional activities from both groups as shown in FIGS. 5A and B. These results indicate that the direction in the combination of elements is less important with respect to the transcriptional activity in synthetic promoters. In both group, most of transcriptional activities were less than 6% of CMV promoter activity. However, some synthetic promoters from both group showed more than two-fold greater activities than that of LPK promoter and more than 8% of CMV promoter activity. To determine whether there was any pattern toward the strong transcriptional activity, selected 6-copy SP-Luc plasmids that showed more than 8% of CMV promoter activity from the 6-copy SP(XE)-Luc group were sequenced. The results are shown in FIG. 4A and indicate that there was no similarity in the order of elements between individual synthetic promoters, and no common pattern in the composition of cis-elements could be found.

Enhancement of the transcriptional activity of synthetic promoters through the addition of 3-copy modules also was studied. The additional modules were inserted at the XhoI/NheI site in selected 6-copy SPXE-Luc plasmids. A total of 17 synthetic promoters from 6-copy SPXE-Luc plasmids with greater than 8% of CMV promoter activity were chosen for further elongation of cis-elements, generating 9-copy SP-Luc plasmids as shown in FIG. 4B. More than 40 clones of 9-copy SP-Luc plasmids were produced from individual 6-copy SPXE-Luc plasmids to cover all possible combinations arising from the addition of 3-copy modules.

The distribution of transcriptional activities of 9-copy SP-Luc plasmids is shown in FIG. 5C. Overall activities of 9-copy SP-Luc plasmids were relatively higher than the original 6-copy SPXE-Luc plasmids. The majority of their activities were less than 12% of CMV promoter activity (FIG. 5C). However, it is notable that a considerable number of clones showed more than 12% of CMV promoter activity, which is greater than the 6-copy SPXE-Luc plasmids, indicating that an increase in the number of cis-elements had a positive effect on transcriptional activity. Furthermore, some clones showed more than 25% of CMV promoter activity that was equivalent to 7 times greater than LPK promoter activity. The composition of cis-elements in these synthetic promoters was examined and shown in FIG. 4B. No similarity in the order of cis-elements among the synthetic promoters was observed.

To investigate synthetic promoter activity in vivo, recombinant adenovirus were generated that express insulin gene under the control of selected 9-copy synthetic promoters. We chose SP23137, SP23142, and 7325 which showed more than 30% of CMV promoter activity. As a ubiquitous and strong viral promoter control, the regulatory region of CMV was used in these experiments. Since the target organ was liver, furin-cleavable proinsulin that could be processed into mature insulin following synthesis was used.

Briefly, recombinant adenovirus was constructed using Transpose-AD™ Adenoviral vector system (Qbiogene, Carlsbad, Calif.) according to the manufacturer's instruction. Furin cleavable rat insulin cDNA (rINSfur) was obtained from VR3503 plasmid that contained rat insulin gene with furin cleavage sites at the B-chain and C-peptide junction (Abai, A. M., Hobart, P. M., & Barnhart, K. M. (1999). Insulin delivery with plasmid DNA. Hum. Gene Ther., 10:2637-2649). rINSfur DNA fragment was digested at XbaI site and cloned into pCR276 adenoviral transfer plasmids, generating pCR276-rINSfur that was used as backbone for further construction of adenoviral vectors containing synthetic promoter. pCMV-rINSfur was generated by insertion of rINSfur region from pCR276-rINSfur into pCR259 adenoviral transfer vector that had CMV promoter to drive transgene expression.

NotI/SalI-digested fragments of SP-rINSfur containing synthetic promoter, fuin cleavable rat insulin cDNA, poly-A region, and SV40 enhancer, were cloned into the PCR276 transfer vector at the same sites, generating PCR276-SP-rINSfur plasmids. The resulting plasmids were transformed into High-Q Transpose-AD™ 294 chemically competent cells, where the transposition of transgene from the recombinant adenovirus. transfer vector to the Transpose-294 plasmid, generating pAd-SP-rINSfur plasmid. pAd-CMV-rINSfur plasmid was generated from pCMV-rINSfur in the same way. DNA extracted from transposition-occurred white colony is re-transformed into chemically competent HighA-1TM cells in order to segregate and amplify the pAd-SP-rINSfur plasmid from Adenovirus transfer vector. The segregated pAd-SP-rINSfur plasmids were then purified into HEK293 cells to generate recombinant adenovirus (rAd-SP-rINSfur) expressing insulin gene under the control of synthetic promoters. rAd-CMV-rINSfur viruses was generated in the same way. Recombinant viruses were amplified in a large scale in HEK293 cells and purified by double CsCl density-gradient centrifugation, and dialyzed against adenovirus dilution buffer (10 mM Tris-Cl pH8.0, 2 mM MgCl2, 4% sucrose). The dialyzed virus was kept at −80° C. for storage. Viral titer was determined by the measurement of O.D. at 260 nm.

Viral administration was performed following streptozotocin (STZ) was administration to NOD/SCID mice via an intraperiotneal injection at a dose of 140 mg/kg body weight in citrate buffer pH 4.0. After blood glucose was increased to 400 mg/dL and continued for 3 consecutive days, recombinant adenoviruses were administered intravenously through tail vein. Blood glucose was measured via the glucose oxidase method, using tail-blood and a One-Touch Profile portable blood glucose monitor (Lifescan, Milpitas Calif.).

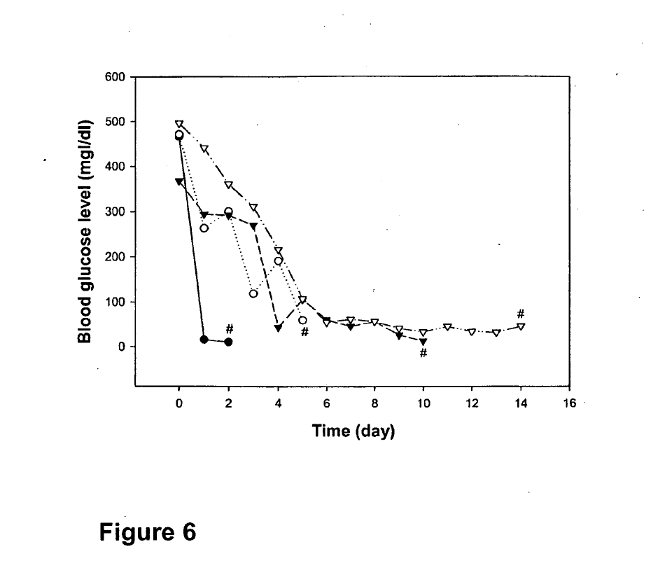

The effects of an uncontrolled, strong promoter to derive insulin gene expression using the CMV promoter was initially examined. A recombinant adenovirus that could produce insulin protein under the control of CMV promoter (rAd-CMV-rINSfur) was generated as described above. These viruses were administered at a titer of 5×10¹⁰ (n=5), 10¹⁰ (n=6), 5×10⁹ (n=4), and 10⁹ (n=4) viral particles into STZ-induced diabetic NOD.scid mice intravenously via tail vein. As shown in FIG. 6, the animals treated with the highest titer of rAd-CMV-rINSfur died within 2 days post-treatment. Although the mice treated with lower titers of viruses survived longer than mice treated with higher titers of viruses, eventually all mice died due to low blood glucose level. All animals suffered severe hypoglycaemia before death as shown in FIG. 6, where the blood glucose levels dropped below 20 mg/dl.

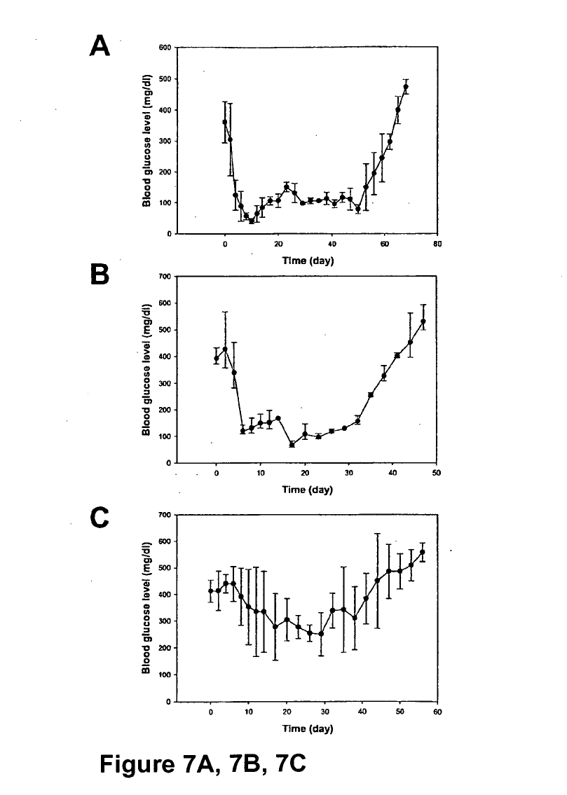

The in vivo activities of 9-copy synthetic promoters that were selected from promoter assay in vitro using a rat hepatoma cell line also were tested. The dose effects of the synthetic promoter were examined through the administration of rAd-23142-rINSfur at different doses of 5×10¹⁰ (n=5), 10¹⁰ (n=6), and 5×10⁹ (n=6) viral particles. The animals treated with highest dose showed a very quick drop in blood glucose levels to a normal range within a few days, while the animals treated with 10¹⁰ viral particles exhibited relatively slow decrease in blood glucose concentration (FIG. 7). After the blood glucose level reached the normal range, normoglycemia was maintained for up to 1 month in mice treated with 10¹⁰ viral particles and longer than 1 month in mice treated with 5×10¹⁰ viral particles. The animals treated with 5×10⁹ viral particles of rAd-23142-rINSfur showed moderately reduced blood glucose levels compared to mice treated with higher doses of virus, indicating that the amount of insulin from this titer of virus was not enough to lower the high blood glucose level into a normal range. As shown in FIG. 7, rAD-23142-rINSfur-treated animals did not show any detrimental hypoglycaemic episodes in contrast to the animals treated with rAD-CMV-rTNSfur. While relatively low blood glucose levels were observed early in mice treated with 5×10¹⁰ viral particles, it did not lead to animal death.

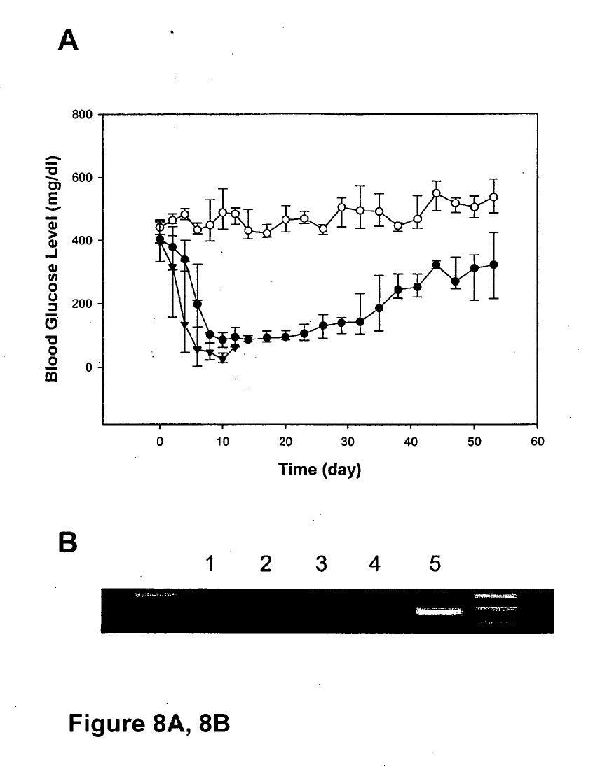

Difference in the transcriptional activity between selected synthetic promoters, SP2313 7 and SP23142, shown in FIG. 4B, that had the same cis-elements for the first 6-copy module and different elements at the later 3-copy module also were examined. Recombinant adenovirus containing SP23137 for insulin gene expression were generated and administered into STZ-induced diabetic NOD.scid mice intravenously via tail vein at a titer of 10 viral particles. As shown in FIG. 8, the treated animals showed normal blood glucose levels within one week, and normoglycemia was preserved up to 1 month similar to rAd-23142-rINSfur-treated animals. There was no significant difference between rAd-23142-rINSfur and rAd-23137-rINSfur-treated animals.

All diabetic NOD.scid mice treated with either rAd-23142-rINSfu or rAd-23137-rINSfur showed recurrence of hyperglycaemia one month following viral treatment. This result can be explained by the occurrence of transient gene expression from first generation adenovirus that deficient only in the EIA gene. Therefore, the presence of adenoviral genome was examined on 10, 25, and 50 days following virus administration. Livers were obtained from rAd-23137-rINSfu-treated NOD.scid mice, isolated genomic DNA, and PCR amplification performed with specific primers for rat insulin gene in adenoviral genome.

Briefly, DNA was purified from the liver of rAd-23137-rINSfur-treated NOD-scid mice on 10, 25, and 50 days after viral administration with DNeasy Tissue kit (Qiagen, Mississauga, ON, Canada) according to the manufacturer's instruction. As positive control, DNA from the liver of rAd-CMV-rINSfur treated animals and pAd-23137-rINSfur (Adenoviral transfer vector containing rat insulin gene) were used. PCR was performed with 100 ng of each DNA with specific primer for rat insulin gene with the same condition described above. The upstream and downstream primers used were as follows: sense, GTGGATGCGCTTCCTG (SEQ ID NO: 17); antisense, ACAATGCCACGCTTCTG (SEQ ID NO:18). After amplification, the products were subjected to electrophoresis on a 1% agarose gel and detected by ethidium bromide staining.

Clear bands were observed at 10 and 25 days, while very faint band were shown in the liver at 50 days, when the animals exhibited a recurred hyperglycaemic state. These results indicated that hyperglycaemic state restored in late period of treatment resulted from the reduced number of adenoviral genome expressing insulin gene in the liver cells.

The glucose responsiveness of the synthetic promoters also was assessed. As shown in FIG. 6, uncontrolled and constitutive expression of the insulin gene caused fatal effects in treated experimental animals, indicating the importance of glucose responsiveness of transcriptional regulatory elements in insulin gene therapy. Although the animals treated with rAd-23137-rfNSfur appeared to be effective in the control of blood glucose levels within normal range, it was beneficial to identify the glucose responsiveness of synthetic promoter. To determine its ability to respond to glucose change, glucose tolerance tests were performed in normal control animals, diabetic control animals, and rAd-23137-rfNSfur-treated animals. After animals were fasted for 16 hr a 200 mM glucose solution was injected at a dose of 2 g/kg body weight into fasted animals intraperitoneally. Blood samples were collected from a small cut at the tip of the mice tail before glucose injection, at 15-min intervals during the first hour, 90 min, 120 min, and 240 min after the glucose load.

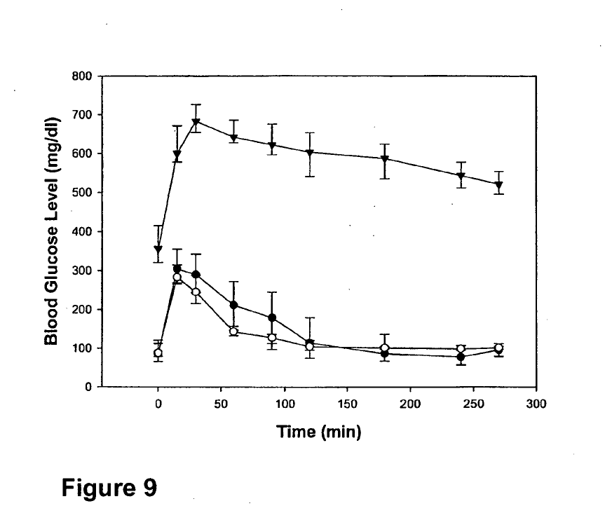

The blood glucose levels prior to glucose challenge were 99±21 mg/dl (normal control mice), 80±21 mg/dl (rAd-23137-rfNSfur-treated mice), and 368±48 mg/dl (diabetic control mice) (FIG. 9). Although the animals treated with rAd-23137-rINSfur showed relatively lower blood glucose levels than normal control mice, they were healthy and did not show hypoglycaemia following the fasting period. Diabetic control mice still suffered from high blood glucose levels. After glucose challenge, blood glucose levels rose to 285±47 mg/dl (rAd-23137-rINSfur-treated mice), 252±36 mg/dl (normal control mice), and 620±48 mg/dl (diabetic control) within 30 min, respectively.

The elevated blood glucose levels decreased quickly to normal range within 60 min in normal control mice, whereas rAd-23137-rfNSfur-treated animals exhibited relatively delayed rates of blood glucose reduction compared to normal control. Even through the treated animals exhibited comparatively lower blood glucose level than normal control mice, no hypoglycaemia was observed in either normal controls or rAd-23137-rINSfur-treated animals.

Liver-specific gene expression by the synthetic promoters also were determined in vitro and in vivo. In this regard, the tissue specificity of the synthetic promoters in vitro was determined by infecting rAd-23137-rfNSfur viruses into several cell lines originated from different species and tissues. rAd-CMV-rfNSfur viruses were used as a control. The source of individual cell lines were as follows: 3T3-L1 cell line from mouse embryonic fibroblast, HeLa cell line from human cervix, L6 cell line from rat skeletal muscle, L-929 cell line from mouse subcutaneous connective tissue, NRK cell line from rat kidney, and H4IIE cell line from rat liver.

Immunological staining of cell lines was performed using the following procedures. Briefly, all cells used in the present study were cultured in Dulbecco's modified Eagle's medium (DMEM) supplemented with 10% fetal bovine serum (Invitrogen), penicillin (2001 U/ml), streptomycin (100 ug/ml), and L-glutamine (2 mM) at 37° C. in a 5% CO2/95% air humidified atmosphere. The cells used were as follows: L6 (Dutheil, N., Shi, F., Dupressoir, T. & Linden, R. M. (2000). Adeno-associated virus site-specifically integrates into a muscle-specific DNA region. Proc. Natl. Acad. Sci. U.S.A., 97:4862-4866). L929 (mouse subcutaneous connective tissue cell line), 3T3 L1 (mouse fibroblast cell line), H4IIE (rat hepatoma cell line), HeLa (human epithelial cell line), and NRK (rat kidney cell line) cells. Cells were seeded onto specifically designed slides (Labtek, Nalge Nucc, Naperville, Ill., USA) at the number of 2×104/well, and incubated for one day, followed by viral infection for additional one day. After the infection, cells were fixed using 4% paraformaldehyde (PFA) in Phosphate buffered saline (PBS) for 15 minute at room temperature, then permeablized with 1% triton X-100 in PBS. Non-specific antibody binding was blocked using blocking buffer (1% (w/v) BSA, 0.2% (v/v) Tween-20 in PBS) for 1 hour. Primary antibody (guinea pig anti-rat insulin (DAKO, Carpiteria, Calif., USA)) was diluted in blocking buffer ( 1/200) and applied to cells for 1 hour. Cells were then washed 3 times in PBS. Secondary antibody (biotinylated anti-guinea pig antibody) was diluted in blocking buffer ( 1/300) and added to cells 1 hour. After 3 washes, HRP-conjugated streptavidin was diluted in blocking buffer ( 1/300) and added to cells for 1 hour, followed by colour developing using Vector VIP (Vector laboratory) according to the manufacturer's instruction.

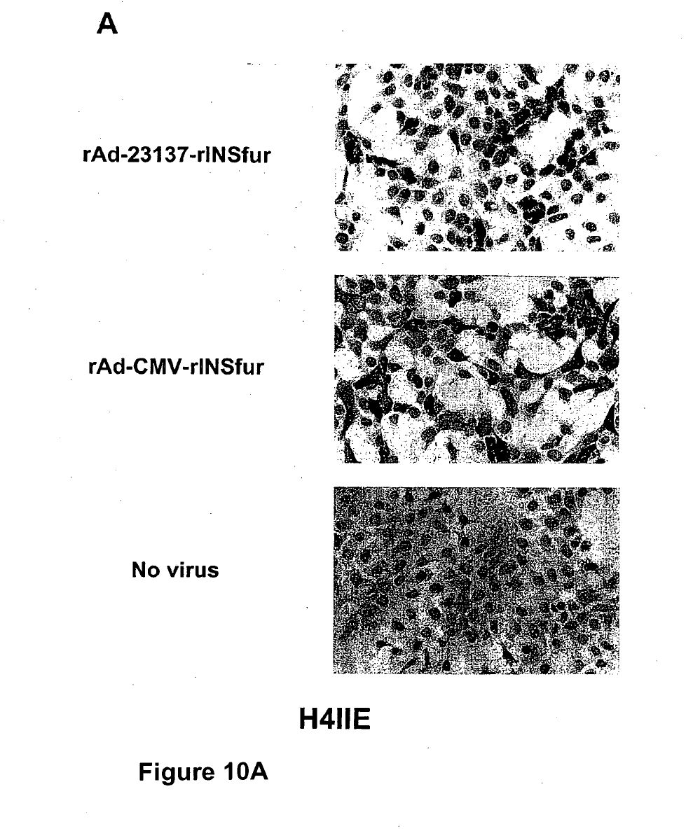

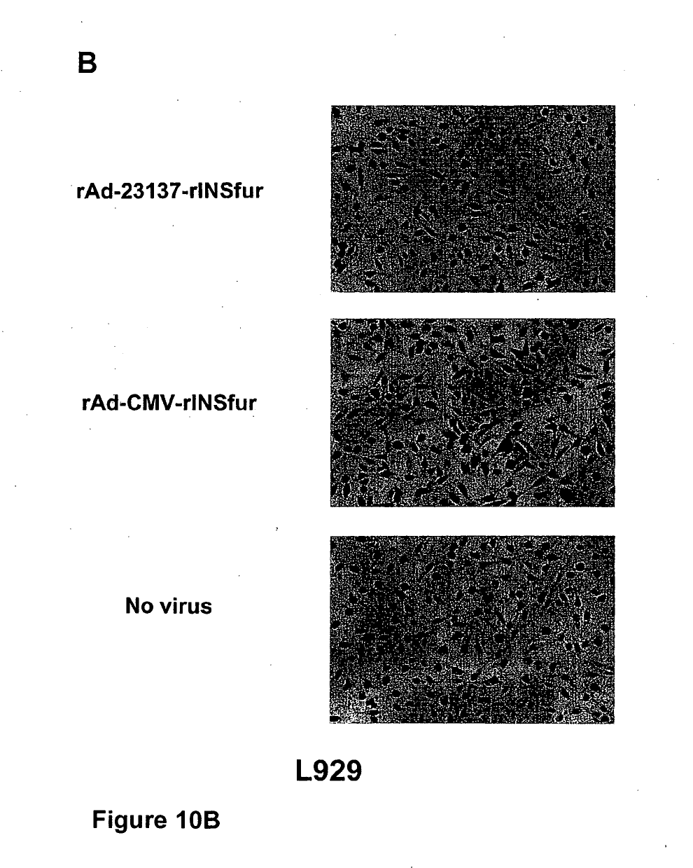

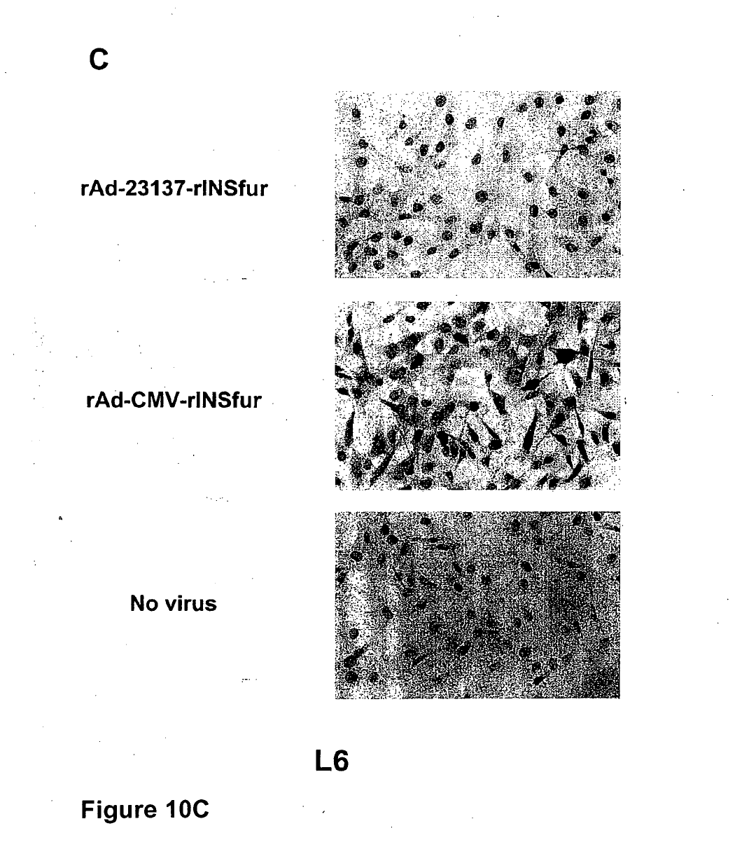

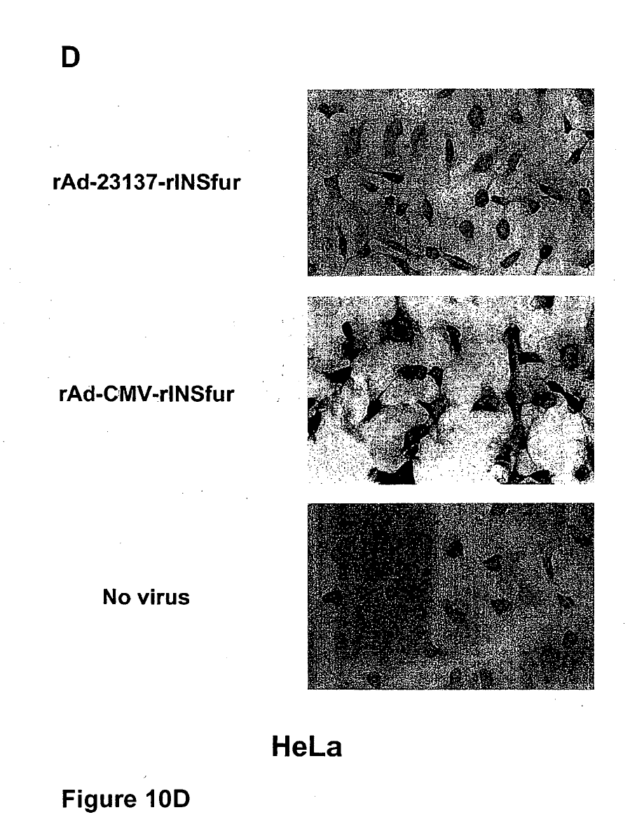

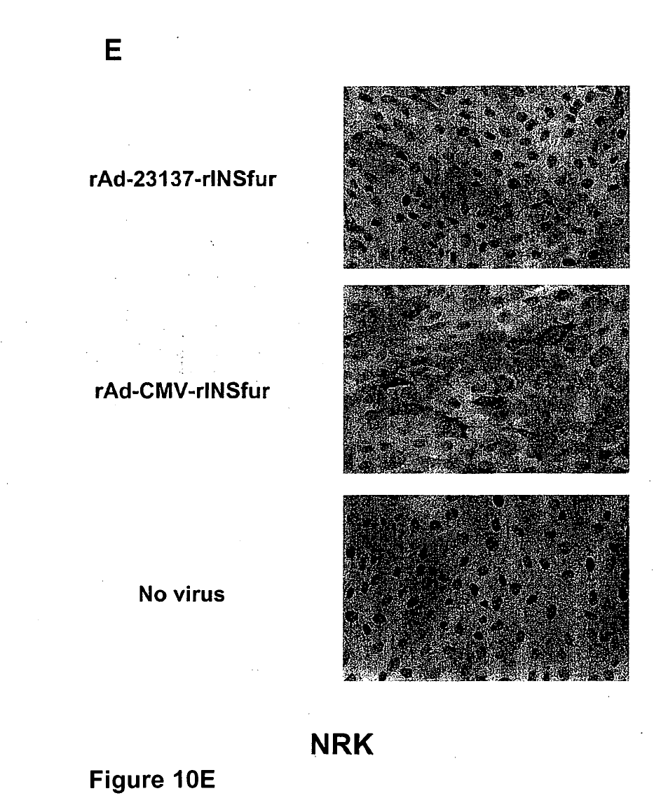

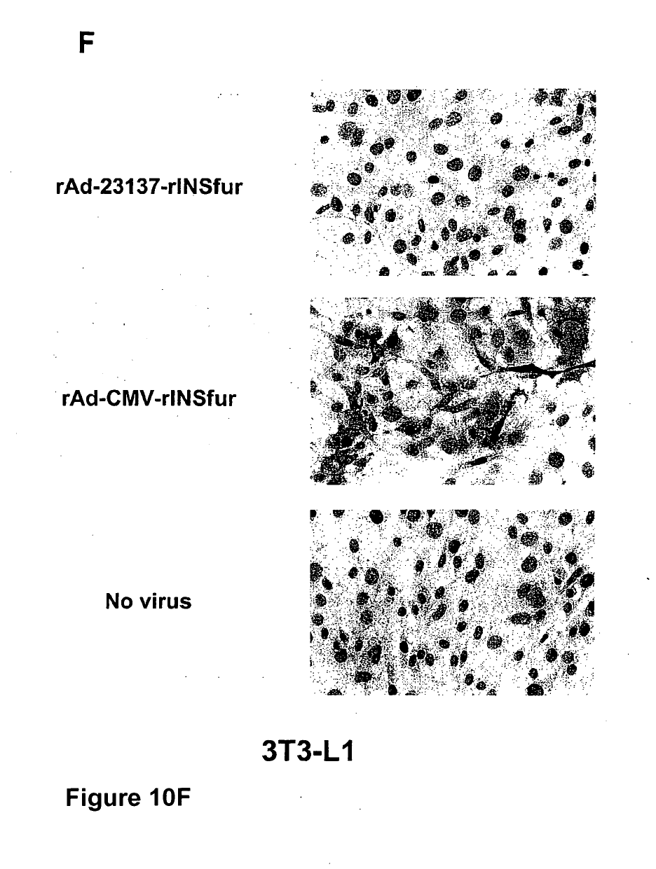

Insulin expression was observed in all cell lines infected with rAd-CMV-rINSfur viruses. The results are shown in FIG. 10 and indicate ubiquitous transcriptional activity of the CMV promoter, whereas SP23137 induced insulin gene expression in H4IIE cells only (FIG. 10A).

The tissue-specific target gene expression by the synthetic promoters also was examinded in the NOD.scid mouse model. For assessment of in vivo activity, rAd-23137-rINSfur viruses were administered into STZ-induced diabetic NOD.scid mice. rAd-CMV-rINSfur viruses were used as ubiquitous control. Upon reaching normoglycemia, treated animals were sacrificed. Several organs such as kidney, spleen, liver, lung, and heart were harvested for total RNA extraction.

Briefly, several organs (liver, spleen, lung, heart, and kidney) of animals were placed in 10 volumes of RNA later RNA stabilization Reagent (Qiagen, Mississauga, ON, Canada), stored at −80° C. Total RNA was isolated using RNeasy Mini Kit (Qiagen, Mississauga, ON, Canada) according to the manufacturer's instruction, and stored at −80° C. 5 ug of total RNA was used to synthesize cDNA using Superscript II reverse transcriptase (Invitrogen, Burlington, ON, Canada) and oligo(dT)12-18 (Invitrogen, Burlington, ON, Canada). PCR was performed using specific primers for rat insulin. Mouse Hypoxanthine phosphoribosyl transferase (HPRT) was used as an internal standard. The upstream and downstream primers used were as follows: rat insulin, sense, GTGGATGCGCTTCCTG (SEQ ID NO:19); rat insulin, antisense, ACAATGCCACGCTTCTG (SEQ ID NO:20); mouse HPRT, sense GTAATGATCAGTCAACGGGGGAC (SEQ ID NO:21); mouse HPRT, antisense CCAGCAAGCTTGCAACCTTAACCA (SEQ ID NO:22). The PCR condition was optimized for each set of primers. The PCR mixture (Chalkley, G. E. & Verrijzer, C. P. (1999). DNA binding site selection by RNA polymerase II TAFs: a TAF(II)250-TAF(II) 150 complex recognizes the initiator. EMBO J, 18:4835-4845) contained 0.2 mM of each deoxynucleotide triphosphate, 1 uM of each specific primer, 1.5 mM or 2 mM MgCl2, 50 mM KCl, 10 mM Tris-Cl, pH 9.0, and 2.5 U of Taq polymerase (NEB). After amplification, the products were subjected to electrophoresis on a 1% agarose gel and detected by ethidium bromide staining.

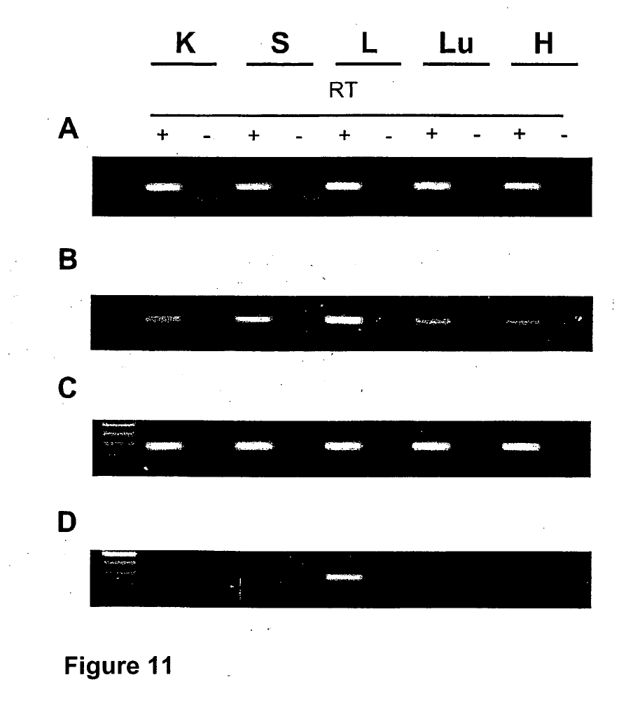

The RT -PCR results are shown in FIG. 11. Rat insulin gene expression could be observed in all organs from rAd-CMV-rINSfur-treated NOD.scid mice due to its ubiquitous transcriptional activity, which was consistent with our in vitro result. However insulin mRNA could be detected only in liver from rAd-23137-rINSfur-treated animals indicating that the synthetic promoter SP23137 had liver-specific transcriptional activity.

In vivo tissue specific expression also was evaluated by histological analysis. The liver, kidney, spleen, and pancreas were removed from virus-treated NOD.scid mice when blood glucose level fell below 150 mg/dL. Glucose (2g/kg body weight) was loaded to boost the insulin expression 2 hours before tissue removal. The samples were fixed with 10% buffered formalin, embedded in paraffin, sectioned at 4.5 μm, and mounted on glass slides. The samples were treated with xylene, and 100%, 90%, 80%, and 70% Ethanol in sequence and washed with tap water. Non-specific antibody binding was blocked using blocking buffer (1% (w/v) BSA, 0.2% (v/v) Tween-20 in PBS) for 1 hour. Primary antibody (guinea pig anti-rat insulin (DAKO, Carpiteria, Calif., USA)) were diluted in blocking buffer ( 1/200) and applied to cells for 1 hour. Cells were then washed 3 times in PBS. Secondary antibody (biotinylated anti-guinea pig antibody) were diluted in blocking buffer ( 1/300) and added to cells 1 hour. After 3 times wash, HRP-conjugated streptavidin was diluted in blocking buffer ( 1/300) and added to cells 1 more hour, followed by colour developing using Vector VIP (Vector laboratory, Burlingame, Calif., USA) according to manufacturer's instruction. After washing with tap water samples were counterstained with Meyer hematoxyln solution. The results obtained demonstrated that the transcriptional activity of the synthetic promoters were limited to the liver and were not observed in other organs.

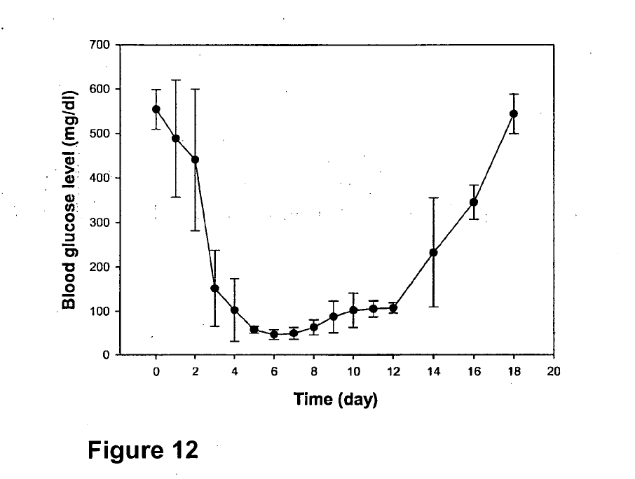

The therapeutic effect of rAd-23137-rfNSfur in a diabetic animal model also was assessed. To test the transcriptional activity of SP23137 in NOD mice, rAd-23137-rINSfur viruses were administered into diabetic NOD mice at a titer of 3×10¹⁰ viral particles. Animals suffered from high blood glucose levels at 554±45 mg/dl prior to viral administration. Blood glucose levels quickly decreased to normal range in 2-3 days as seen in FIG. 12. These results demonstrate that rAd-23137-rINSfur has therapeutic effects in the autoimmune type 1 diabetic animal model that is immunocompetent, as well as in the chemically-induced diabetic animal model that is immunodeficient. However, Normoglycemia in rAd-23137-treated NOD mice was maintained for 10 days and then hyperglycaemia recurred. The recurrence of hyperglycaemia was observed in NOD.scid mice in 1 month following normalization of blood glucose level. This earlier recurrence of hyperglycaemia in NOD mice might result from a more intensive host immune response to adenoviral vector.

The results of the above studies revealed that most of the synthetic promoters with 3 element copies had comparable transcriptional activities to natural LPK promoter, whereas multimerized single elements had relatively low activity, which was consistent with previous reports (Li, X., Eastman, E. M., Schwartz, R. J., & Draghia-Akli, R. (1999). Synthetic muscle promoters: activities exceeding natually occurrng regulatory sequences. Nat. Biotechnol., 17:241-245). However, it also has been shown recently that the inclusion of additional transcription factor binding elements within a construct can enhance gene expression (Walters, M. C., Fiering, S., Eidemiler, J., Magis, W., Groudine, M., & Martin, D. J. (1995). Enhancers increase the probability but not the level of gene expression. Proc. Natl. Acad. Sci. U.S.A., 92:7125-7129; Sutherland, H. G., Martin, D. & Whitelaw, E. (1997). A globin enhancer acts by increasing the proportion of erythrocytes expressing a linked trans gene. Mol. Cell Biol., 17:1607-1.614). Therefore, enhancement of the transcriptional activities of 3 copy synthetic promoters was investigated through introduction of additional 3-copy modules in the constructs.

To investigate the orientation effects, 3-copy modules were inserted in the same or opposite direction with pre-existing modules. The distribution of transcriptional activity of these constructs indicated that the majority of them showed the same or less transcriptional activities compared to 3-copy synthetic promoters (FIG. 5A and 5B). However, some of the higher copy number promoters showed much higher activity (FIG. 3B). There was no significant difference in the distribution of transcriptional activities between the constructs with the same or opposite direction of the 3-copy modules. These overall observations indicate that some combinations of cis-elements can have a synergistic effect on the promoter activity. However, the presence of common patterns in synthetic promoters that showed over 8% of CMV promoter activity was not observed with either the 6 or 9 copy modules. The distribution of transcriptional activities in 9-copy synthetic promoters showed that most of them had the same or less activity whereas some had superior activity compared to their parental constructs. No similarity in the arrangement sequence of individual elements at 9-copy synthetic promoters with high activity was found.

Even though the transcriptional activity of synthetic promoters was enhanced compared to natural LPK promoter, the glucose responsiveness also was assessed for their ability to regulate insulin gene therapy. As shown it FIG. 6, uncontrolled expression of insulin by CMV promoter in rAd-CMV-rINSfur-treated animals resulted in death due to hypoglycaemia. In contrast, insulin expression by synthetic promoter was appropriately controlled in response to glucose change in the STZ-induced diabetic model. It was notable that no severe hypoglycaemia was found in animals treated with 5×10¹⁰ viral particles. The vector dose was not increased further in order to avoid viral toxicity of the first generation adenovirus. In addition, fasting blood glucose levels in rAd-SP-rINSfur-treated animals was comparable to that in normal healthy animals, indicating that insulin expression is regulated in response to blood glucose levels. These results indicate that the synthetic promoters of the invention have the ability to regulate insulin production in response to the change of glucose level and maintain normoglycemia in diabetic animals.

Moreover, the results from glucose tolerance tests in rAd-23137-rINSfur-treated animals support the ability of synthetic promoters to respond to glucose change for insulin expression. After a peak at 15 and 30 min post glucose challenge, blood glucose levels dropped to normal ranges similar to control. However, control animals showed normal glucose within 60 min, whereas rAd-23137-rINSfur-treated animals showed the same level at 120 min following glucose loading. Moreover, relatively low blood glucose level was observed in treated animals compared with normal control animals between 180 and 240 min after glucose challenge, and it went back to similar range with normal control animals at 270 min. These results indicate that the synthetic promoters of the invention still can be further optimized, especially with respect to negative feedback. However, the synthetic promoters are much more efficient than previously reported promoters, where non-fasting glucose level in the treated animals were moderately high. Negative regulation of the synthetic promoters can be achieved by introduction of insulin responsive element which has been shown to repress promoter activity in the presence of insulin (O'brien, R. M., Streeper, R. S., Ayala, J. E., Stadelmaier, B. T., & Homnbuckle, L. A. (2001). Insulin-regulated gene expression. Biochem. Soc. Trans., 29:552-558). In addition, it also is possible to control the half-life of transgenic insulin mRNA by incorporating specific elements into its sequence that result in rapid degradation (Wilusz, C. J. & Wilusz, J. (2004). Bringing the role of mRNA decay in the control of gene expression into focus. Trends Genet., 20:491-497), as the potential risk of insulin-induced hypoglycaemia depends largely on the relative stability of insulin mRNA (Dong, H. & Woo, S. L. (2001). Hepatic insulin production for type 1 diabetes. Trends Endocrinol. Metab., 12:441-446).

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. An isolated tissue specific glucose responsive promoter comprising a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE).

2. The promoter of claim 1, wherein said polymerase binding domain comprises a liver pyruvate kinase (LPK) promoter.

3. The promoter of claim 1, wherein said HNF-1 element comprises about 25-30 nucleotides.

4. The promoter of claim 3, wherein said HNF-1 element comprises SEQ ID NO:1.

5. The promoter of claim 1, wherein said C/EBP response element comprises about 20-25 nucleotides.

6. The promoter of claim 5, wherein said C/EBP response element comprises SEQID NO:2.

7. The promoter of claim 1, wherein said GRE comprises about 45-50 nucleotides.

8. The promoter of claim 7, wherein said GRE comprises SEQ ID NO:3.

9. The promoter of claim 1, wherein said HNF-1 element, said C/EBP response element and said GRE of within said tripartite transcription factor binding cis element are substantially adjacent.

10. The promoter of claim 1, wherein said HNF-1 element, said C/EBP response element and said GRE of within said tripartite transcription factor binding cis element are contiguous.

11. The promoter of claim 1, comprising a tripartite transcription factor binding cis element selected from the elements shown in FIG. 3A.

12. The promoter of claim 1, further comprising a second tripartite transcription factor binding cis element.

13. The promoter of claim 12, comprising a tripartite transcription factor binding cis element selected from the elements shown in FIG. 3B or FIG. 4A.

14. The promoter of claim 1, further comprising a third tripartite transcription factor binding cis element.

15. The promoter of claim 14, comprising a tripartite transcription factor binding cis element selected from the elements shown in FIG. 4B.

16. The promoter of claim 1, further comprising an operationally linked insulin encoding nucleic acid or a subunit coding sequence.

17. A host cell comprising the tissue specific glucose responsive promoter of claim 1, 12 or 14.

18. A method of treating or preventing diabetes comprising administering to an individual an effective amount of a viral particle having a vector comprising a tissue specific glucose responsive promoter comprising a polymerase binding domain 3′ to at least one tripartite transcription factor binding cis element having a hepatocyte nuclear factor-1 (HNF-1) element, a CAAT/enhancer binding protein (C/EBP) response element and a glucose-response element (GRE) operationally linked to an insulin encoding nucleic acid, wherein expression of said insulin encoding nucleic acid is tissue specific and glucose responsive.

19. The tissue specific glucose responsive promoter of claim 18, wherein said polymerase binding domain comprises a liver pyruvate kinase (LPK) promoter.

20. The tissue specific glucose responsive of claim 18, wherein said HNF-1 element comprises about 25-30 nucleotides.

21. The tissue specific glucose responsive promoter of claim 20, wherein said HNF-1 element comprises SEQ ID NO:1.

22. The tissue specific glucose responsive promoter of claim 18, wherein said C/EBP response element comprises about 20-25 nucleotides.

23. The tissue specific glucose responsive promoter of claim 22, wherein said C/EBP response element comprises SEQ ID NO:2.

24. The tissue specific glucose responsive promoter of claim 18, wherein said GRE comprises about 45-50 nucleotides.

25. The tissue specific glucose responsive promoter of claim 24, wherein said GRE comprises SEQ ID NO:3.

26. The tissue specific glucose responsive promoter of claim 18, wherein said HNF-1 element, said C/EBP response element and said GRE of within said tripartite transcription factor binding cis element are substantially adjacent.

27. The tissue specific glucose responsive promoter of claim 18, wherein said HNF-1 element, said C/EBP response element and said GRE of within said tripartite transcription factor binding cis element are contiguous.

28. The tissue specific glucose responsive promoter of claim 18, comprising a tripartite transcription factor binding cis element selected from the elements shown in FIG. 3A.

29. The tissue specific glucose responsive promoter of claim 18, further comprising a second tripartite transcription factor binding cis element.

30. The tissue specific glucose responsive promoter of claim 29, comprising a tripartite transcription factor binding cis element selected from the elements shown in FIG. 3B or FIG. 4A.

31. The tissue specific glucose responsive promoter of claim 18, further comprising a third tripartite transcription factor binding cis element.

32. The tissue specific glucose responsive promoter of claim 31, comprising a tripartite transcription factor binding cis element selected from the elements shown in FIG. 4B.

33. The method of claim 18, wherein said vector comprises an adenoviral vector.

34. The method of claim 18, wherein said viral particle is administered in a pharmaceutically acceptable carrier.


SEQUENCE LISTING (22 sequences)

SEQ ID NO: 1 - DNA, 27 bp, Artificial Sequence
  synthetic oligonucleotide fragment
ctagctggttaatgattaaccaggact

SEQ ID NO: 2 - DNA, 22 bp, Artificial Sequence
  synthetic oligonucleotide fragment
tcgcaagttgcgcaatatcgcg

SEQ ID NO: 3 - DNA, 48 bp, Artificial Sequence
  synthetic oligonucleotide fragment
gggcgcacggggcactcccgtggttcctggactctggcccccagtgta

SEQ ID NO: 4 - DNA, 27 bp, Artificial Sequence
  synthetic oligonucleotide fragment
ctagctggttaatgattaaccaggact

SEQ ID NO: 5 - DNA, 22 bp, Artificial Sequence
  synthetic oligonucleotide fragment
tcgcaagttgcgcaatatcgcg

SEQ ID NO: 6 - DNA, 48 bp, Artificial Sequence
  synthetic oligonucleotide fragment
tacactgggggccagagtccaggaaccacgggagtgccccgtgcgccc

SEQ ID NO: 7 - DNA, 96 bp, Homo sapiens
  CDS (1)...(96)
tttgtgaaccaacacctgtgcggctcacacctggtggaagctctctacctagtgtgcggggaacgaggcttcttctacacacccaagacccgccgg

SEQ ID NO: 8 - protein, 32 aa, Homo sapiens
FVNQHLCGSHLVEALYLVCGERGFFYTPKTRR

SEQ ID NO: 9 - DNA, 66 bp, Homo sapiens
  CDS (1)...(63)
ggcattgtggaacaatgctgtaccagcatctgctccctctaccagctggagaactactgcaactag

SEQ ID NO: 10 - protein, 21 aa, Homo sapiens
GIVEQCCTSICSLYQLENYCN

SEQ ID NO: 11 - DNA, 17 bp, Rattus sp.
catgtgaaaacgtcgtg

SEQ ID NO: 12 - DNA, 17 bp, Mus musculus
cacgtggtggccctgtg

SEQ ID NO: 13 - DNA, 17 bp, Rattus sp.
cacgctggagtcagccc

SEQ ID NO: 14 - DNA, 17 bp, Rattus sp.
cacggggcactcccgtg

SEQ ID NO: 15 - DNA, 17 bp, Rattus sp.
catgtgccacaggcgtg

SEQ ID NO: 16 - protein, 4 aa, Artificial Sequence
  synthetic cleavage site
  VARIANT (1)...(4): Xaa = Any Amino Acid
RXKR

SEQ ID NO: 17 - DNA, 16 bp, Artificial Sequence
  PCR primer
gtggatgcgcttcctg

SEQ ID NO: 18 - DNA, 17 bp, Artificial Sequence
  PCR primer
acaatgccacgcttctg

SEQ ID NO: 19 - DNA, 16 bp, Rattus sp.
gtggatgcgcttcctg

SEQ ID NO: 20 - DNA, 17 bp, Rattus sp.
acaatgccacgcttctg

SEQ ID NO: 21 - DNA, 23 bp, Mus musculus
gtaatgatcagtcaacgggggac

SEQ ID NO: 22 - DNA, 24 bp, Mus musculus
ccagcaagcttgcaaccttaacca